(19) United States
(12) Patent Application Publication
(10) Pub. No.: US 2025/0375582 A1
(43) Pub. Date: Dec. 11, 2025

(54) DRY POWDER INHALERS

(71) Applicant: HANGZHOU XIXI PHARMACEUTICALS, Hangzhou (CN)

(72) Inventors: Lan CHEN, Hangzhou (CN); Donghao CHEN, Hangzhou (CN); Hui CHEN, Hangzhou (CN)

(73) Assignee: HANGZHOU XIXI PHARMACEUTICALS, Hangzhou (CN)

(21) Appl. No.: 19/300,646

(22) Filed: Aug. 14, 2025

Related U.S. Application Data
(63) Continuation-in-part of application No. PCT/CN2024/073652, filed on Jan. 23, 2024.

(30) Foreign Application Priority Data
Feb. 14, 2023 (CN) 202310108306.8
Feb. 14, 2023 (CN) 202310108307.2
Feb. 14, 2023 (CN) 202320213730.4

(51) Int. Cl. A61M 15/00

(52) U.S. Cl. CPC A61M 15/0035; A61M 15/0041

(57) ABSTRACT

A dry powder inhaler is provided, including an upper structure, a lower structure, and a piercing base. The upper structure undergoes a longitudinal linear movement relative to the lower structure, the upper structure is formed with a capsule chamber and a piercing channel arranged laterally outside of the capsule chamber, one or more piercing needles undergo a lateral piercing movement along the piercing channel, a drive structure is provided among the upper structure, the lower structure, and the piercing structure. When the upper structure undergoes a longitudinal linear movement relative to the lower structure, the drive structure drives the piercing needles to move laterally along the piercing channel, thereby piercing a capsule positioned in the capsule chamber. The dry powder inhaler enables the capsule to be pierced from the side along the piercing channel by pressing the lower structure and the upper structure.

### CROSS-REFERENCE TO RELATED APPLICATIONS

This application is a continuation-in-part application of International Application No. PCT/CN2024/073652 filed on Jan. 23, 2024, which claims priority to Chinese Patent Application No. 202320213730.4 filed on Feb. 14, 2023, Chinese Patent Application No. 202310108306.8 filed on Feb. 14, 2023, and Chinese Patent Application No. 202310108307.2 filed on Feb. 14, 2023, the entire contents of each of which are hereby incorporated by reference.

### TECHNICAL FIELD

The present disclosure relates to the field of medical devices, and in particular, to a dry powder inhaler.

### BACKGROUND

Dry powder inhaler (DPI), as a new type of inhaler developed after the metered-dose inhaler (MDI) without propellants, it utilizes the patient's inspiratory airflow to drive the drug powder into the airway, with 10% to 30% of the drug deposited in the lower respiratory tract, which is slightly higher than the MDI. It is typically indicated to all patients over 4 years old who can cooperate with the inspiratory, with a broad demographic applicability.

Currently, there are three types of dry powder inhalers: blister-type, capsule-based and reservoir inhalers. Capsule-based inhalers can be divided into pen-shaped and elliptical grip-type inhalers, both of them have a piercing structure, especially for the elliptical grip-type inhalers. The piercing structure of the existing capsule-based dry powder inhaler is typically positioned on the bottom side, or on one side or both sides of the bottom. Take the inhaler CN215083633U and the piercing structure of the powder inhaler CN105920709B as example, regardless of whether the configuration is single-sided or double-sided, the piercing structure comprises a lateral elastic actuation mechanism connected to piercing needles. The piercing needles are driven by the elastic actuation mechanism to pierce the capsule from its lateral aspect, with the actuation direction of the elastic actuation mechanism aligned with the movement direction of the piercing needles.

However, there are some technical problems in the capsule-based dry powder inhaler with elastic actuation mechanism and piercing structure. 1) The setting of the lateral-pressed elastic actuation mechanism requires the coordinated lateral actuation action between the index finger and thumb, which is not conducive to effective force application, presenting significant operational challenges for patients with reduced hand strength in utilizing the inhaler. 2) In addition, the setting of lateral elastic actuation mechanism of the inhaler results in a large bilateral width, affecting the overall aesthetics. The large horizontal distance is also one of the factors that increase operational complexity.

The objective of this invention is to overcome the technical deficiencies associated with the alignment of actuation direction of the elastic actuation mechanism and the movement direction of the piercing needles, particularly the requirement for excessive operating force and the wide horizontal width.

### SUMMARY

Embodiments of the present disclosure provide a dry powder inhaler. The dry powder inhaler comprises: a piercing base, the piercing base being provided with one or more piercing needles for piercing a capsule from a side; an upper structure, the upper structure being formed with a capsule chamber and a piercing channel, wherein the capsule chamber is configured for accommodating the capsule; the piercing channel is arranged laterally outside of the capsule chamber and connected to the capsule chamber, and the piercing needles undergo a lateral piercing movement along the piercing channel; a lower structure for accommodating a portion of the upper structure, the upper structure being subjected to a longitudinal linear movement relative to the lower structure; and a drive structure arranged on at least one of the upper structure and the lower structure, the piercing base being driven by the drive structure to undergo movement. The drive structure moves with the upper structure and the lower structure undergoing a longitudinal linear relative approach movement, the drive structure drives the piercing base to move in a direction of the piercing channel, the piercing needles move with the piercing base along the piercing channel to pierce the capsule in the capsule chamber; the drive structure moves with the upper structure and the lower structure undergoing a longitudinal linear relative separation movement, the drive structure drives the piercing base to move in the direction of the piercing channel, and the piercing needles leave the capsule chamber with the piercing base along the piercing channel. The piercing channel is a straight channel and the piercing needles are straight needles; and the piercing needles are contained in a portion of the piercing channel at any time. The drive structure includes an inclined track arranged within the lower structure for a tilting movement of the piercing base, the inclined track being tilted in a direction gradually inclined from a position at an upper edge of the lower structure to a lower portion of the lower structure and toward the capsule chamber, when the upper structure and the lower structure are subjected to the longitudinal linear relative approach movement, the piercing base is driven by the inclined track and guided by the piercing channel to move toward the capsule chamber, and the piercing needles enter the capsule chamber along the piercing channel to complete piercing of the capsule.

Embodiments of the present disclosure also provide another dry powder inhaler. The dry powder inhaler comprises: a piercing base, the piercing base being provided with one or more piercing needles for piercing a capsule from a side; an upper structure, the upper structure being formed with a capsule chamber and a piercing channel, wherein the capsule chamber is configured for accommodating the capsule; the piercing channel is arranged laterally outside of the capsule chamber and connected to the capsule chamber, and the piercing needles undergo a lateral piercing movement along the piercing channel; a lower structure for accommodating a portion of the upper structure, the upper structure being subjected to a longitudinal linear movement relative to the lower structure; and a drive structure arranged on at least one of the upper structure and the lower structure, the piercing base being driven by the drive structure to undergo movement. The drive structure moves with the upper structure and the lower structure undergoing a longitudinal linear relative approach movement, the drive structure drives the piercing base to move in a direction of the piercing channel, the piercing needles move with the piercing base along the piercing channel to pierce the capsule in the capsule chamber; the drive structure moves with the upper structure and the lower structure undergoing a longitudinal linear relative separation movement, the drive structure drives the piercing base to move in the direction of the piercing channel, and the piercing needles leave the capsule chamber with the piercing base along the piercing channel. The piercing channel is a straight channel and the piercing needles are straight needles; and the piercing needles are contained in a portion of the piercing channel at any time. The drive structure includes a linkage structure connected to the piercing base and a hinge structure; and the hinge structure is configured to articulate the linkage structure with the lower structure and the piercing base. The linkage structure includes at least one main connecting rod obliquely connected to the piercing base, one end of the main connecting rod is hinged to a sidewall of the lower structure through the hinge structure, and the other end of the main connecting rod is hinged to the piercing base through the hinge structure. The main connecting rod is tilted in a direction gradually tilting upwardly from a position at the upper edge of the lower structure and toward the capsule chamber; and when the upper structure and the lower structure undergo the longitudinal linear relative movement, the main connecting rod rotates with the relative longitudinal linear movement, the main connecting rod rotates to drive the piercing base to move in the direction of the piercing channel, and the piercing needles enter the capsule chamber along the piercing channel with the piercing base to pierce the capsule in the capsule chamber.

### BRIEF DESCRIPTION OF THE DRAWINGS

The present disclosure is further described in terms of exemplary embodiments. These exemplary embodiments are described in detail with reference to the drawings. These embodiments are non-limiting embodiments, in which like reference numerals represent similar structures throughout the drawings, wherein:

FIG. 1 is a schematic diagram illustrating an overall structure of a dry powder inhaler without a protective cap according to some embodiments of the present disclosure;

FIG. 2 is a schematic diagram illustrating a front-to-rear longitudinal cross-sectional structure of an overall structure of an anti-separation structure with a limit rod according to some embodiments of the present disclosure;

FIG. 3 is a schematic diagram illustrating a portion of an upper structure and a lower structure according to some embodiments of the present disclosure;

FIG. 4 is a schematic diagram illustrating a structure of a groove-type inclined track embodiment in an unpierced state according to some embodiments of the present disclosure;

FIG. 5 is a schematic diagram illustrating a structure of a groove-type inclined track embodiment in a pierced state according to some embodiments of the present disclosure;

FIG. 6 is a schematic diagram illustrating a partial longitudinal cross-sectional structure of the upper structure and the lower structure of the groove-type inclined track embodiment according to some embodiments of the present disclosure;

FIG. 7 is a schematic diagram illustrating an overall longitudinal cross-sectional structure of the dry powder inhaler without a protective cap according to some embodiments of the present disclosure;

FIG. 8 is a schematic diagram illustrating an internal structure of a single-side piercing type of the groove-type inclined track embodiment according to some embodiments of the present disclosure;

FIG. 9 is a schematic diagram illustrating a structure of a piercing base of the groove-type inclined track embodiment according to some embodiments of the present disclosure;

FIG. 10 is a schematic diagram illustrating an upper structure and a piercing base of the groove-type inclined track embodiment according to some embodiments of the present disclosure;

FIG. 11 is a schematic diagram illustrating an upper structure of the groove-type inclined track embodiment according to some embodiments of the present disclosure;

FIG. 12 is a schematic diagram illustrating a left-right longitudinal cross-sectional structure of a lower structure according to some embodiments of the present disclosure;

FIG. 13 is a schematic structural diagram illustrating an upper structure and a lower structure of a slope-type inclined track embodiment, with the upper structure not showing a finger placement area according to some embodiments of the present disclosure;

FIG. 14 is a schematic diagram illustrating a left-right longitudinal cross-sectional structure of an upper structure and a lower structure of the slope-type inclined track embodiment, with the upper structure not showing the finger placement area according to some embodiments of the present disclosure;

FIG. 15 is a schematic diagram illustrating a left-right longitudinal cross-sectional structure of the slope-type inclined track embodiment in an unpierced state according to some embodiments of the present disclosure;

FIG. 16 is a schematic diagram illustrating a left-right longitudinal cross-sectional structure of the slope-type inclined track embodiment in a pierced state according to some embodiments of the present disclosure;

FIG. 17 is a schematic diagram illustrating a left-right longitudinal cross-sectional structure of the lower structure of the slope-type inclined track embodiment according to some embodiments of the present disclosure;

FIG. 18 is a schematic diagram illustrating a structure of a piercing base with parallel rods of the slope-type inclined track embodiment according to some embodiments of the present disclosure;

FIG. 19 is a schematic diagram illustrating a partially enlarged structure of a T-shaped limiting groove of the slope-type inclined track embodiment according to some embodiments of the present disclosure;

FIG. 20 is a schematic diagram illustrating a piercing base with T-shaped protrusions of the slope-type inclined track embodiment according to some embodiments of the present disclosure;

FIG. 21 is a schematic diagram illustrating a structure of an upper structure and a lower structure of a tilting main connecting rod embodiment, with the upper structure not showing a finger placement area according to some embodiments of the present disclosure;

FIG. 22 is a schematic diagram illustrating a left-right longitudinal cross-sectional structure of an upper structure and a lower structure of the tilting main connecting rod embodiment, with the upper structure not showing the finger placement area according to some embodiments of the present disclosure;

FIG. 23 is a schematic diagram illustrating a left-right longitudinal cross-sectional structure of the tilting main connecting rod embodiment in an unpierced state according to some embodiments of the present disclosure;

FIG. 24 is a schematic diagram illustrating a left-right longitudinal cross-sectional structure of the tilting main connecting rod embodiment in a pierced state according to some embodiments of the present disclosure;

FIG. 25 is a schematic diagram illustrating a relationship between an incline angle of the main connecting rod, a length of the main connecting rod, a piercing distance of piercing needles, and a longitudinal linear movement distance between the upper structure and the lower structure according to some embodiments of the present disclosure;

FIG. 26 is a schematic diagram illustrating a structure of the upper structure, a linkage structure, and the piercing needles in a combined state of the tilting main connecting rod embodiment according to some embodiments of the present disclosure;

FIG. 27 is a schematic diagram illustrating a structure of the linkage structure and the piercing needles in a combined state of the tilting main connecting rod embodiment according to some embodiments of the present disclosure;

FIG. 28 is a schematic diagram illustrating a structure of an inhalation channel section according to some embodiments of the present disclosure;

FIG. 29 is a schematic diagram illustrating a structure of the nozzle section according to some embodiments of the present disclosure; and

FIG. 30 is a schematic diagram illustrating a left-right longitudinal cross-sectional structure of the slope-type inclined track embodiment in an alternative pierced state according to some embodiments of the present disclosure.

In the figures, 1, upper structure; 111, capsule chamber; 1111, weight sensor; 112, rotary chamber; 1121, airflow channel opening; 1122, flow velocity sensor; 12, piercing channel; 13, controller; 2, lower structure; 21, accommodating chamber; 22, pressure sensor; 23, vibration device; 3, piercing needle; 31, needle holder; 4, reset structure; 40, reset spring; 41, upper lumen; 42, lower lumen; 51, limit rod; 52, locking protrusion; 53, movement groove; 54, limit protrusion; 55, guide groove; 56, guide surface; 571, direction-guiding groove; 572, direction-guiding column; 61, operation table; 611, anti-slip pattern; 612, anastomosis table; 613, finger placement structure; 62, inhalation channel section; 621, columnar channel tube; 63, nozzle section; 631, anastomosis tube; 64, protective cap; 7, inclined track; 71, groove-type inclined track; 711, guide opening; 72, piercing base; 720, motion-guiding structure; 721, protruding column; 722, cylindrical body; 723, columnar cavity; 724, secondary adaptation groove; 725, secondary adaptation protrusion; 730, adapting portion; 81, slope-type inclined track; 811, inclined surface; 812, anastomosing surface; 82, main base; 831, spring structure; 832, parallel lumen; 833, parallel rod; 841, T-shaped limiting groove; 842, T-shaped protrusion; 851, bottom structure; 852, bottom cavity; 853, secondary adaptation cavity; 854, secondary adaptation rod; 91, main connecting rod; 921, running channel; 922, plate structure; 93, maintaining connecting rod; 94, connecting portion of connecting rod.

### DETAILED DESCRIPTION

The technical solutions in the embodiments of the present disclosure are hereinafter described clearly and completely by means of particular specific embodiments, and it is obvious that the described embodiments are only a part of the embodiments of the present disclosure and not all of them, and a person skilled in the art can easily understand other advantages and efficacies of the present disclosure by the contents disclosed in the present disclosure. The present disclosure may also be implemented or applied in different other specific embodiments, and the following embodiments and features in the embodiments may be combined with each other in a way that does not conflict with each other, and based on the embodiments of the present disclosure, those skilled in the art may, without making any creative efforts, realize the other embodiments belonging to the scope of protection of the present disclosure.

Embodiments of the present disclosure provide a dry powder inhaler (hereinafter referred to as the inhaler). The inhaler includes a piercing base, an upper structure, a lower structure, and a drive structure. The piercing base is provided with one or more piercing needles for piercing a capsule from a side. The upper structure is formed with a capsule chamber and a piercing channel, the capsule chamber is configured for accommodating the capsule, the piercing channel is arranged laterally outside of the capsule chamber and connected to the capsule chamber, and the piercing needles undergo a lateral piercing movement along the piercing channel. The lower structure is configured for accommodating a portion of the upper structure, the upper structure is subjected to a longitudinal linear movement relative to the lower structure. The drive structure is arranged on at least one of the upper structure and the lower structure, and the piercing base is driven by the drive structure to undergo movement. The drive structure moves with the upper structure and the lower structure undergoing a longitudinal linear relative approach movement, the drive structure drives the piercing base to move in a direction of the piercing channel, the piercing needles move with the piercing base along the piercing channel to pierce the capsule in the capsule chamber. The drive structure moves with the upper structure and the lower structure undergoing a longitudinal linear relative separation movement, the drive structure drives the piercing base to move in the direction of the piercing channel, and the piercing needles leave the capsule chamber with the piercing base along the piercing channel. The piercing channel is a straight channel and the piercing needles are straight needles. The piercing needles are contained in a portion of the piercing channel at any time. The drive structure includes an inclined track arranged within the lower structure for a tilting movement of the piercing base, the inclined track is tilted in a direction gradually inclined from a position at an upper edge of the lower structure to a lower portion of the lower structure and toward the capsule chamber, when the upper structure and the lower structure are subjected to the longitudinal linear relative approach movement, the piercing base is driven by the inclined track and guided by the piercing channel to move toward the capsule chamber, and the piercing needles enter the capsule chamber along the piercing channel to complete piercing of the capsule.

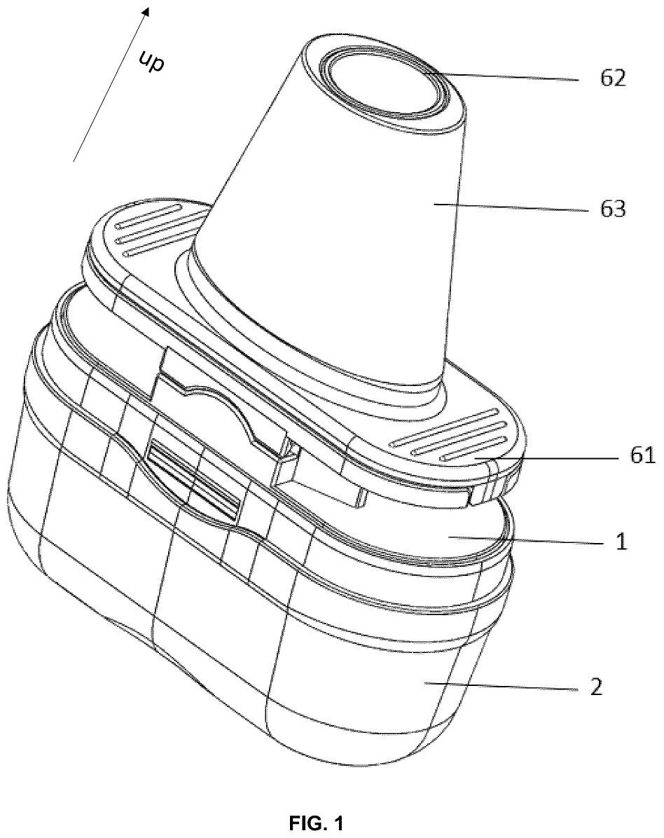

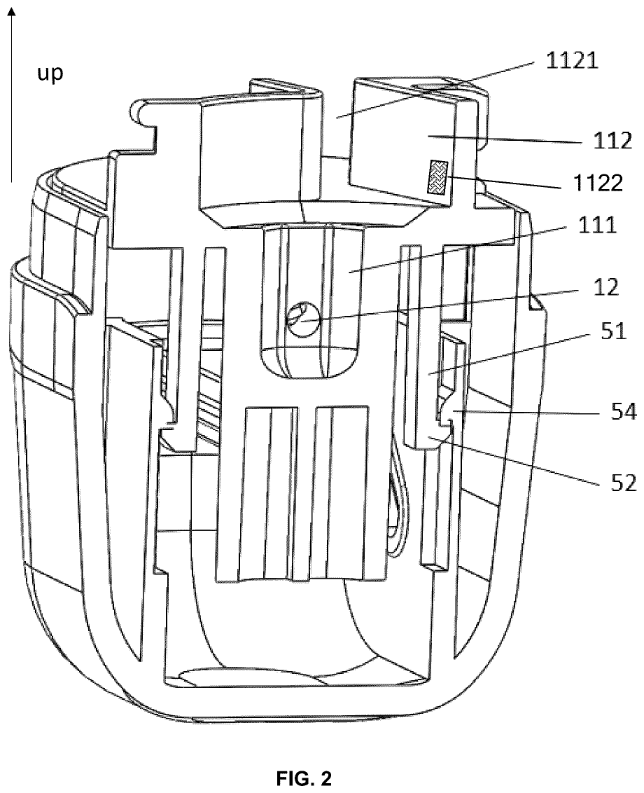

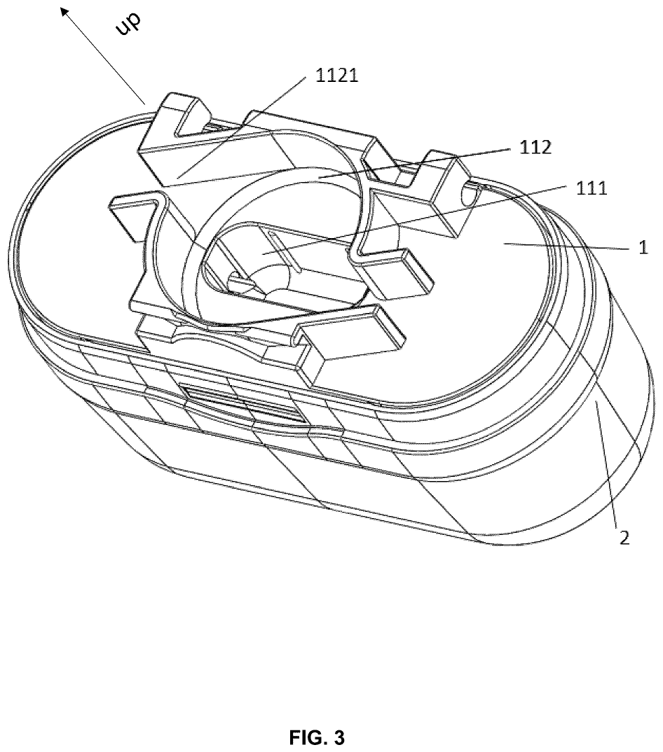

FIG. 1 is a schematic diagram illustrating an overall structure of a dry powder inhaler without a protective cap according to some embodiments of the present disclosure; FIG. 2 is a schematic diagram illustrating a front-to-rear longitudinal cross-sectional structure of an overall structure of an anti-separation structure with a limit rod according to some embodiments of the present disclosure; and FIG. 3 is a schematic diagram illustrating a portion of an upper structure and a lower structure according to some embodiments of the present disclosure.

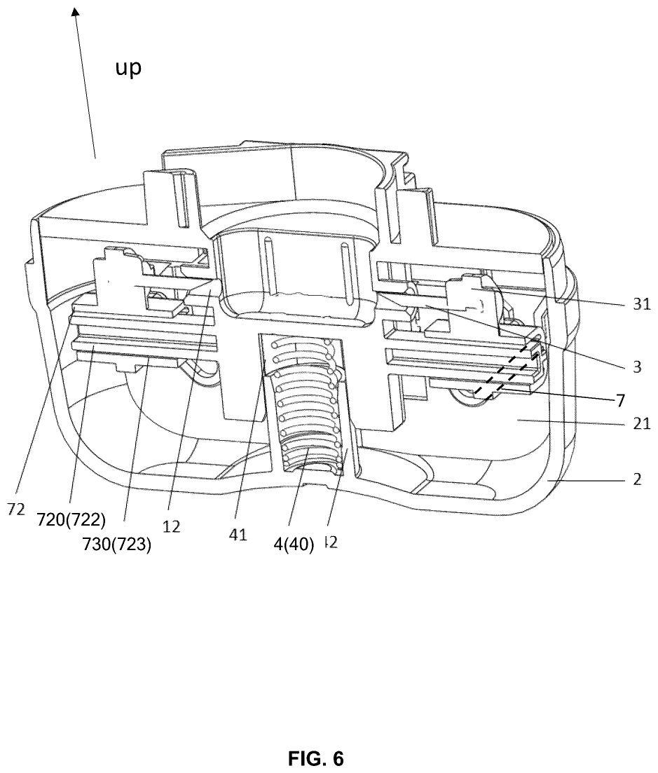

In some embodiments, as shown in FIGS. 1-3, the inhaler includes an upper structure 1, a lower structure 2, and a piercing base 72 (see FIG. 6). The piercing base 72 refers to a base for fixing other piercing structures, and the piercing base 72 includes one or more piercing needles 3 (see FIG. 6). The upper structure 1 refers to a portion of a structure located above the inhaler, and the upper structure 1 is formed with a capsule chamber 111 and a piercing channel 12 on the outside of the capsule chamber 111 (see FIG. 6). The piercing channel 12 provides a piercing trajectory for the piercing needles 3 to undergo a lateral piercing movement, i.e., the piercing needles 3 undergo the lateral piercing movement along the piercing channel 12. The lower structure 2 refers to a portion of a structure located below the inhaler, and the upper structure 1 undergoes a longitudinal linear relative movement with the lower structure 2, which simultaneously drives the piercing base 72 to pierce the capsule in the capsule chamber 111 from the piercing channel 12. The piercing channel 12 is oriented in a horizontal direction or an inclined upward or an inclined downward direction to enable piercing of the capsule to be accomplished from a horizontal side, an upper side, or a lower side. The inhaler is further provided with a drive structure, the drive structure is a structure for driving the movement of the piercing base 72, which is provided on the upper structure 1 and/or the lower structure 2; and the piercing base 72 is driven by the drive structure to undergo movement.

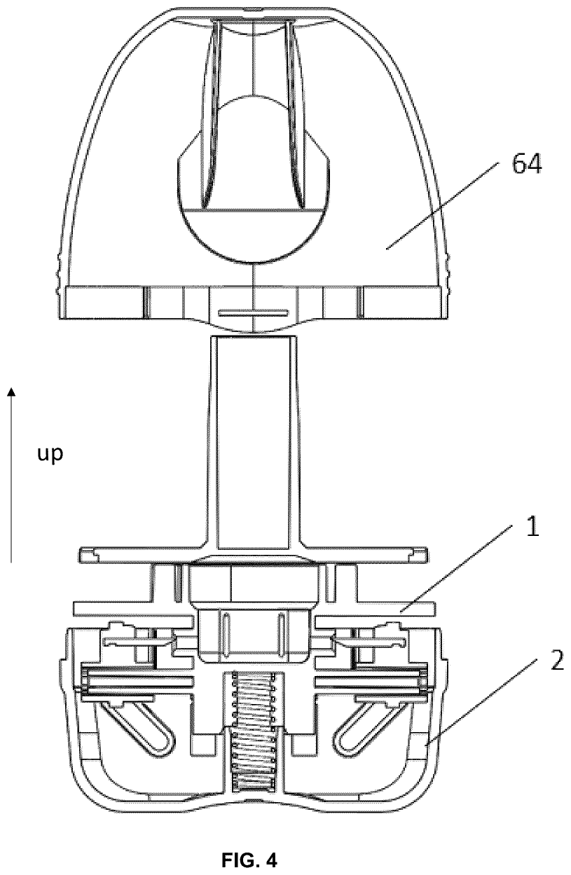

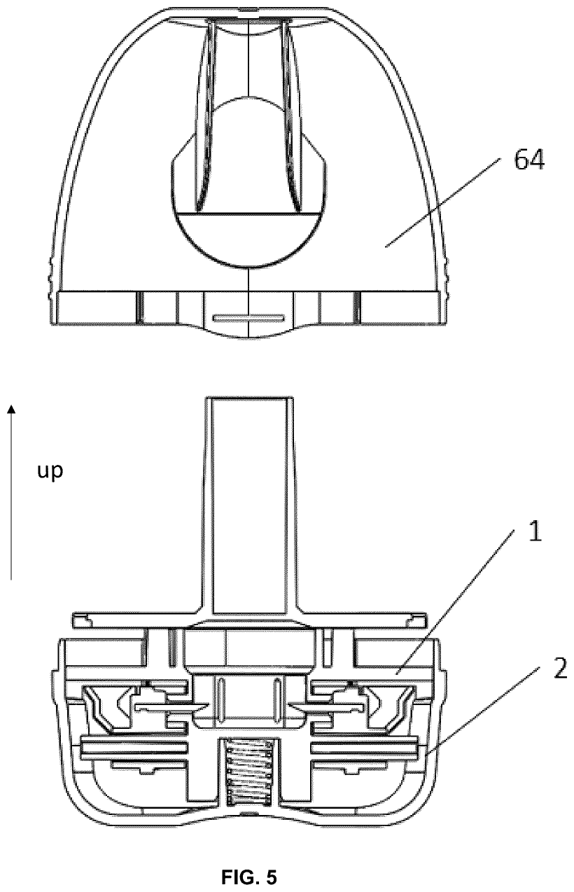

FIG. 4 is a schematic diagram illustrating a structure of a groove-type inclined track embodiment in an unpierced state according to some embodiments of the present disclosure; and FIG. 5 is a schematic diagram illustrating a structure of a groove-type inclined track embodiment in a pierced state according to some embodiments of the present disclosure.

In some embodiments, the drive structure moves with the longitudinal linear relative approach movement of the upper structure 1 and the lower structure 2, while the drive structure drives the piercing base 72 to move in the direction of the piercing channel 12, and the piercing needles 3 move along the piercing channel 12 with the piercing base 72 into the capsule chamber 111 to pierce the capsule in the capsule chamber 111. The drive structure moves with the longitudinal linear relative separation movement of the upper structure 1 and the lower structure 2, and the drive structure drives the piercing base 72 to move in the direction of the piercing channel 12, and the piercing needles 3 leaves the capsule chamber 111 along the piercing channel 12 with the piercing base 72, as shown in FIGS. 1-5.

FIG. 6 is a schematic diagram illustrating a partial longitudinal cross-sectional structure of the upper structure and the lower structure of the groove-type inclined track embodiment according to some embodiments of the present disclosure.

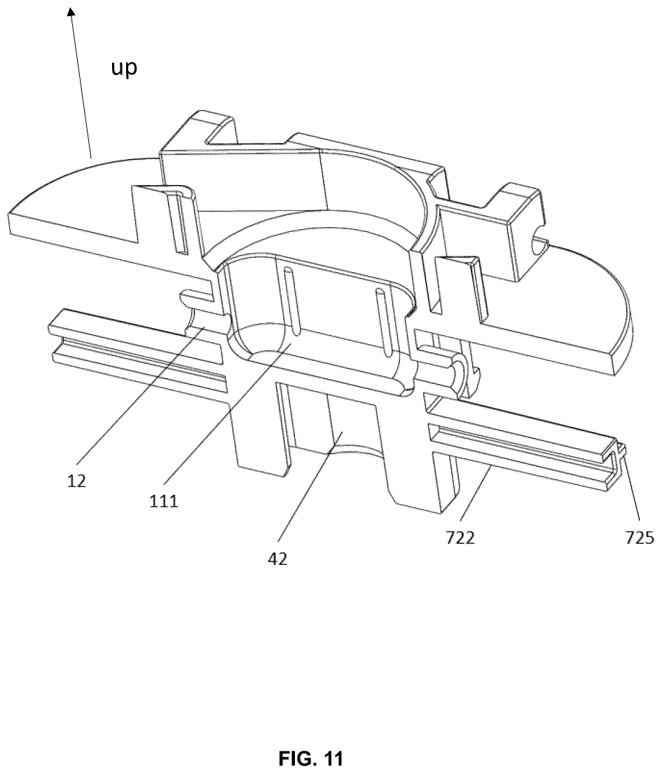

In some embodiments, shapes and a movement relationship of the lower structure 2 and the upper structure 1 may be as follows, the lower structure 2 is formed with an accommodating chamber 21, the accommodating chamber 21 is configured to hold the upper structure 1 and the piercing base 72, and the upper structure 1 undergoes the longitudinal linear relative movement within the accommodating chamber 21 of the lower structure 2. The upper structure 1 further includes a rotary chamber 112 disposed above the capsule chamber 111, and inclined airflow channel openings 1121 are disposed on both sides of the rotary chamber 112. In some embodiments, a reset structure 4 is provided at the bottom of the upper structure 1 and the lower structure 2 for realizing a motion reset of the upper structure 1 and the lower structure 2. In some embodiments, when the inhaler includes a locking protrusion 52 and a limit protrusion 54, the reset structure 4 may bring the locking protrusion 52 into contact with the limit protrusion 54 in a state where the inhaler is not in use. In some embodiments, the reset structure 4 is a reset spring 40, as shown in FIGS. 1-3 and 6. More descriptions regarding the locking protrusion 52 and the limit protrusion 54 may be found in FIGS. 2 and 11 and the related descriptions thereof.

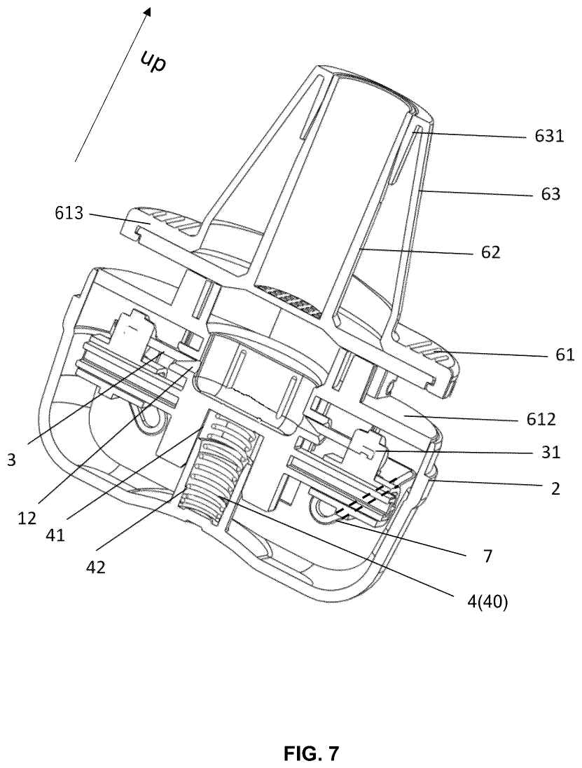

FIG. 7 is a schematic diagram illustrating an overall longitudinal cross-sectional structure of the dry powder inhaler without a protective cap according to some embodiments of the present disclosure.

In some embodiments, the piercing channel 12 is a straight channel and the piercing needles 3 are straight needles; the piercing needles 3 are contained in a portion of the piercing channel 12 at any time when the upper structure 1 undergoes the longitudinal linear movement with the lower structure 2. In some embodiments, as shown in FIGS. 6 and 7, the drive structure includes an inclined track 7 provided within the lower structure 2 for realizing a tilting movement of the piercing base 72, and the inclined track 7 is tilted in a direction gradually inclined from a position at the upper edge of the lower structure 2 to the lower portion of the lower structure 2 and towards the capsule chamber 111, which can realize that when the upper structure 1 and the lower structure 2 undergo the longitudinal linear relative approach movement, the piercing base 72 moves towards the capsule chamber 111 under the driving effect of the inclined track 7 and the guiding effect of the piercing channel 12, and the piercing needles 3 enter into the capsule chamber 111 along the piercing channel 12 to complete the piercing of the capsule.

In some embodiments, the piercing channel 12 is oriented in a horizontal direction or an inclined upward or an inclined downward direction. A direction of the piercing movement of the piercing channel 12 and a direction of the longitudinal linear movement of the upper structure 1 and the lower structure 2 may be combined into a direction of combined movement along the inclined track 7. This arrangement can effectively ensure that various settings of the inclined track 7 are possible.

In some embodiments, the combined movement distance of the piercing distance of the piercing base 72 in the piercing channel 12 and the longitudinal linear movement distance is a tilting movement distance on the inclined track. In some embodiments, after an incline angle of the inclined track 7 is determined, a proportional relationship between the piercing distance and the longitudinal linear movement distance of the upper structure 1 and the lower structure 2 is determined based on the incline angle of the inclined track 7. Alternatively, the incline angle is calculated based on the desired longitudinal linear movement distance and the piercing distance of the piercing channel 12. This approach enables a more rapid determination of various parameters of the various structures in the inhaler, see FIG. 6.

Taking the direction of the piercing channel 12 being horizontal as an example, a length of the inclined track is L1, a piercing distance of the piercing needles is L2, and a longitudinal linear movement distance between the upper structure and the lower structure is L3, and a relationship between the length L1, the piercing distance L2, and the longitudinal linear movement distance L3 is represented that the length L1 is greater than a length of a hypotenuse of a right triangle formed by L2 and L3.

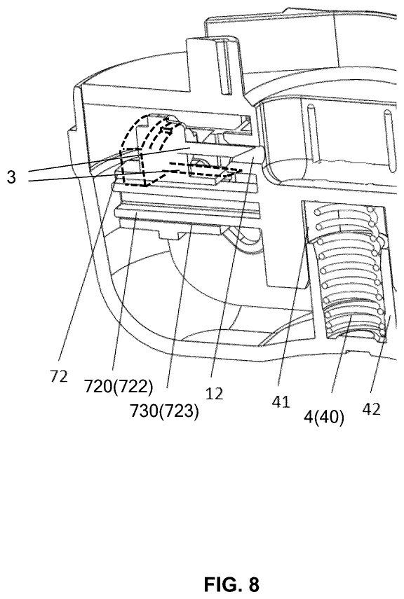

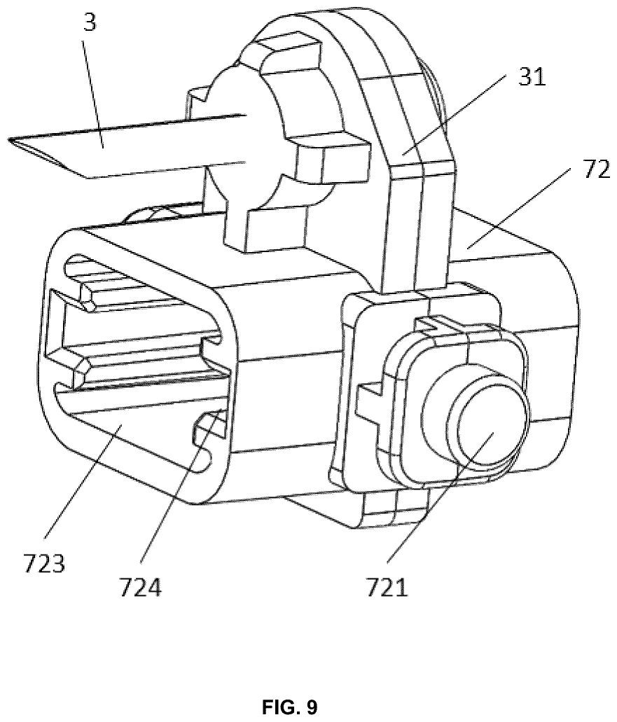

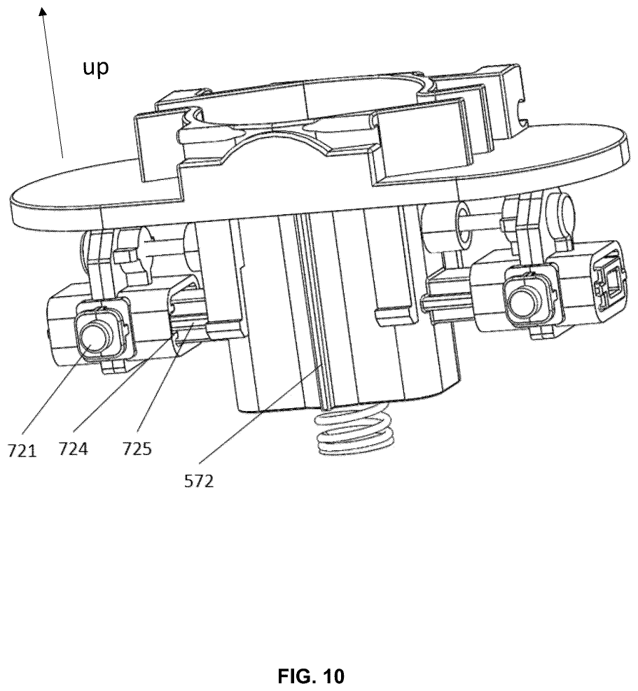

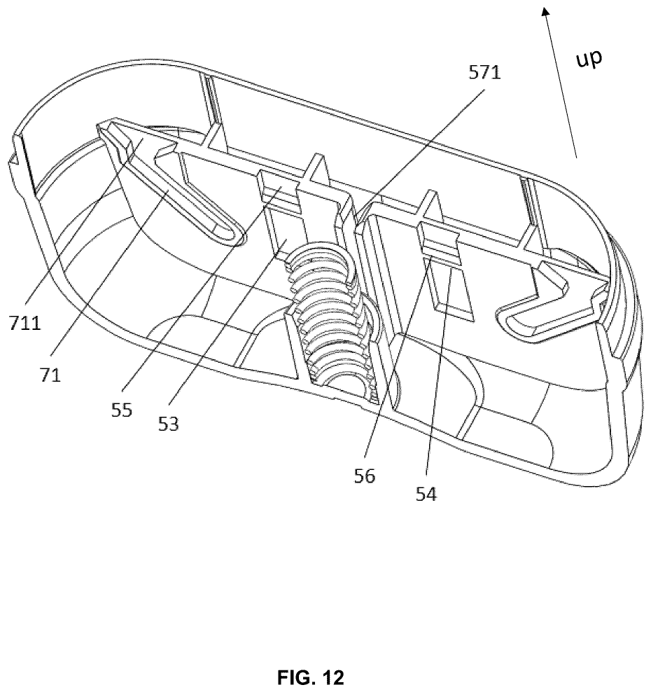

FIG. 8 is a schematic diagram illustrating an internal structure of a single-side piercing type of the groove-type inclined track embodiment according to some embodiments of the present disclosure. FIG. 9 is a schematic diagram illustrating the structure of a piercing base of the groove-type inclined track embodiment according to some embodiments of the present disclosure. FIG. 10 is a schematic diagram illustrating an upper structure and a piercing base of the groove-type inclined track embodiment according to some embodiments of the present disclosure. FIG. 11 is a schematic diagram illustrating the upper structure of the groove-type inclined track embodiment according to some embodiments of the present disclosure. FIG. 12 is a schematic diagram illustrating a left-right longitudinal cross-sectional structure of the lower structure according to some embodiments of the present disclosure.

In some embodiments, as shown in FIGS. 9-12, the inclined track 7 is a groove-type inclined track 71 provided on the front and rear sidewalls of the lower structure 2, and a protruding column 721 extending into the groove-type inclined track 71 is provided on the piercing base 72. In some embodiments, a groove-type opening of the groove-type inclined track 71 is a square or “T” shaped opening. In some embodiments, one or two piercing bases 72 are provided around the capsule chamber 111.

In some embodiments, when the inhaler is a single-side piercing type, one piercing base 72 is provided, the front and rear sidewalls of the lower structure 2 are provided with two groove-type inclined tracks 71, the front and rear sides of the piercing base 72 are provided with protruding columns 721 that extend into the groove-type inclined tracks 71, and the piercing base 72 is provided with two parallel piercing needles 3, and two parallel piercing channels 12 are correspondingly provided on the same side of the inhaler, referring to FIG. 8.

In some embodiments, referring to FIGS. 5-7 and 10-12, when the inhaler is a double-side piercing type, two piercing bases 72 are provided, and four groove-type inclined tracks 71 are provided correspondingly on the front and rear sidewalls of the lower structure 2, the front and rear sides of each of the piercing bases 72 are provided with protruding columns 721 that extend into the groove-type inclined tracks 71. Exemplarily, the two piercing bases 72 and the four groove-type inclined tracks 71 are symmetrically provided around the capsule chamber 111, one piercing needle 3 is provided on one piercing base 72, and two piercing channels 12 arranged in parallel or in the same straight line are provided on both sides of the inhaler.

In some embodiments, with reference to FIGS. 6 and 7, the inhaler is further provided with a motion-guiding structure 720 for ensuring movement of the piercing base 72 along the piercing channel 12, the piercing base 72 is provided with an adapting portion 730 in combination with the motion-guiding structure 720, and the adapting portion 730 moves on the motion-guiding structure 720. In some embodiments, the motion-guiding structure 720 is a cylindrical body 722 or a columnar cavity protruding from the left and right sidewalls of the upper structure 1 or the lower structure 2, and the motion-guiding structure 720 is oriented in a direction that is parallel to the piercing channel 12. Exemplarily, the motion-guiding structure 720 is the cylindrical body 722 or the columnar cavity extending from the sidewall of the upper structure 1.

In some embodiments, with reference to FIGS. 6 and 10, the motion-guiding structure 720 is adapted with the adapting portion 730 in such a manner as follows, when the motion-guiding structure 720 is a cylindrical body 722, the adapting portion 730 is the columnar cavity 723 that is socketed to the outside of the cylindrical body 722, and the protruding column 721 protrudes outwardly from the front and rear walls of the columnar cavity 723. In other embodiments, when the motion-guiding structure 720 is a columnar cavity, the adapting portion 730 is a cylindrical body disposed inside the columnar cavity, the protruding column extends out from the outside of the front and rear walls of the cylindrical body, and the transverse penetration notch is arranged on the front and rear sidewalls of the columnar cavity to realize the protrusion of the protruding column and ensure the protruding movement.

In some embodiments, a secondary adaptation groove 724 is provided within the columnar cavity 723 and a secondary adaptation protrusion 725 is provided on the cylindrical body 722. In some embodiments, the secondary adaptation groove 724 is symmetrically disposed on the inner sides of the front and rear sidewalls or on the inner sides of the top and bottom sidewalls of the columnar cavity 723. This arrangement enables better adaptation of the motion-guiding structure 720 to the adapting portion 730.

In some embodiments, the piercing base 72 is provided with a needle holder 31 accommodating the piercing needles 3, and the piercing needles 3 are integrally connected to the needle holder 31 or the piercing needles 3 are combined with the needle holder 31, and the position remains unchanged after the combination, so as to better set the piercing needles 3 on the piercing base 72. A positional relationship between the needle holder 31 and the motion-guiding structure 720 may be set as follows, the needle holder 31 is set above the motion-guiding structure 720 and the needle holder 31 is smaller in size than the motion-guiding structure 720, which ensures the stabilization of the structure of the piercing base 72.

In some embodiments, a guide opening 711 is provided at the uppermost part of the lower structure 2, the guide opening 711 is larger in size than the inclined track opening, and the guide opening 711 is a triangular opening, so as to conveniently realize the combination of a columnar base into the groove-type inclined track 71, referring to FIGS. 6, 10, and 12. The columnar base refers to a structure formed by combining the motion-guiding structure 720 with the piercing base 72.

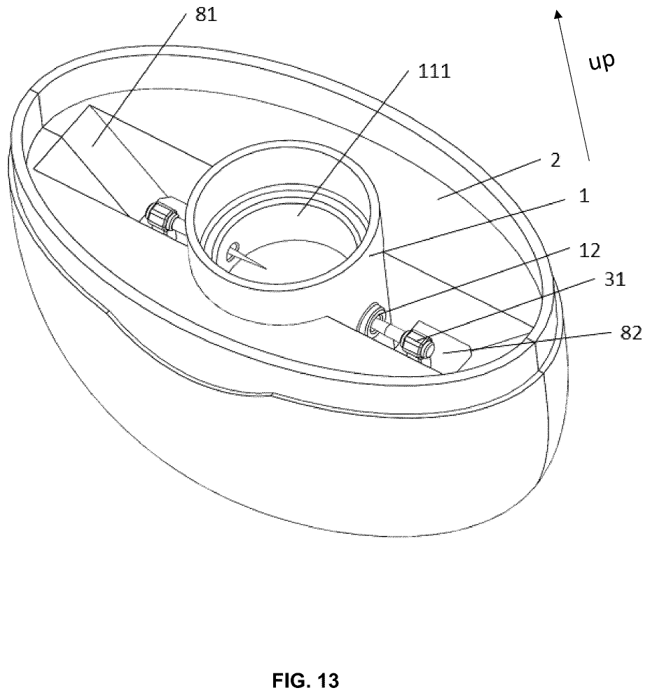

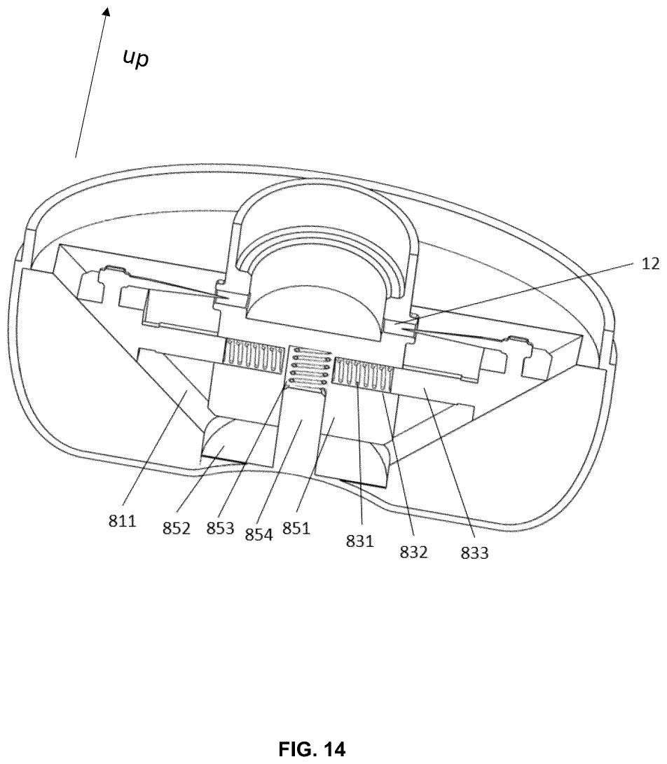

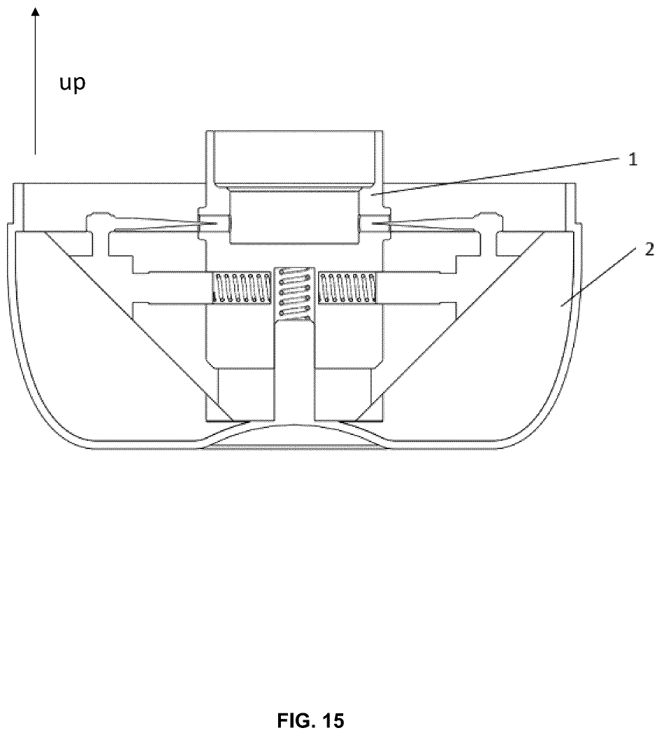

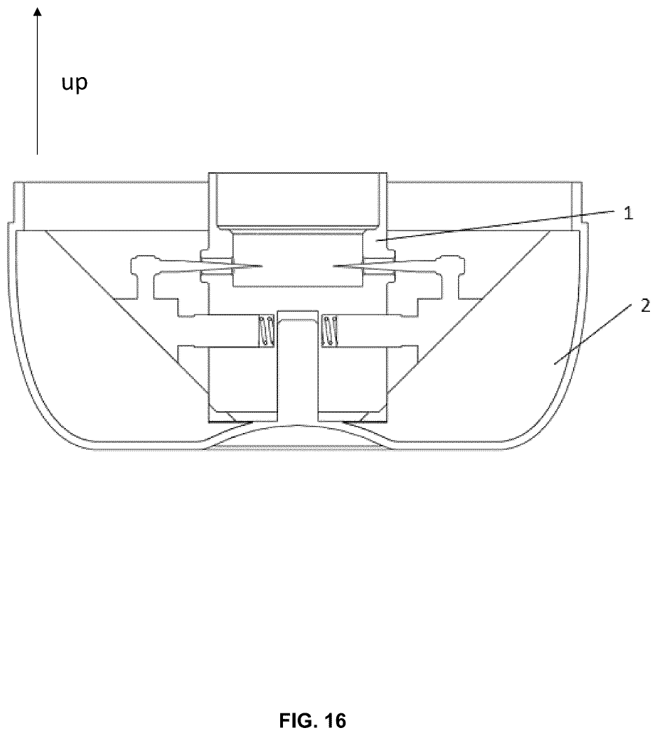

FIG. 13 is a schematic structural diagram illustrating an upper structure and a lower structure of an of a slope-type inclined track embodiment, with the upper structure not showing a finger placement area according to some embodiments of the present disclosure; FIG. 14 is a schematic diagram illustrating a left-right longitudinal cross-sectional view of an upper structure and a lower structure of the slope-type inclined track embodiment, with the upper structure not showing the finger placement structure according to some embodiments of the present disclosure; FIG. 15 is a schematic diagram illustrating a left-right longitudinal cross-sectional structure of the slope-type inclined track embodiment in an unpierced state according to some embodiments of the present disclosure; and FIG. 16 is a schematic diagram illustrating a left-right longitudinal cross-sectional structure of the slope-type inclined track embodiment in a pierced state according to some embodiments of the present disclosure.

In some embodiments, as shown in FIGS. 13-16, the inclined track may also be a slope-type inclined track 81 provided on the left and right sidewalls of the lower structure 2, and the movement track of the slope-type inclined track 81 is an inclined surface 811 of the slope-type inclined track 81, and the piercing base 72 is provided with an anastomosing surface 812 that fits with the inclined surface 811 of the slope-type inclined track 81.

In some embodiments, when the inhaler is of the single-side piercing type, one slope-type inclined track 81 and one piercing base 72 are provided, and two parallel piercing needles 3 are provided on the one piercing base 72, and two parallel piercing channels 12 are provided on the same side of the inhaler. In some embodiments, when the inhaler is a double-side piercing type, two slope-type inclined tracks 81 are symmetrically provided on the left and the right sidewalls of the lower structure 2 and two piercing bases 72 are correspondingly provided, and one piercing needle 3 is provided on one piercing base 72, and two piercing channels 12 arranged in parallel or in the same straight line are provided on both sides of the inhaler.

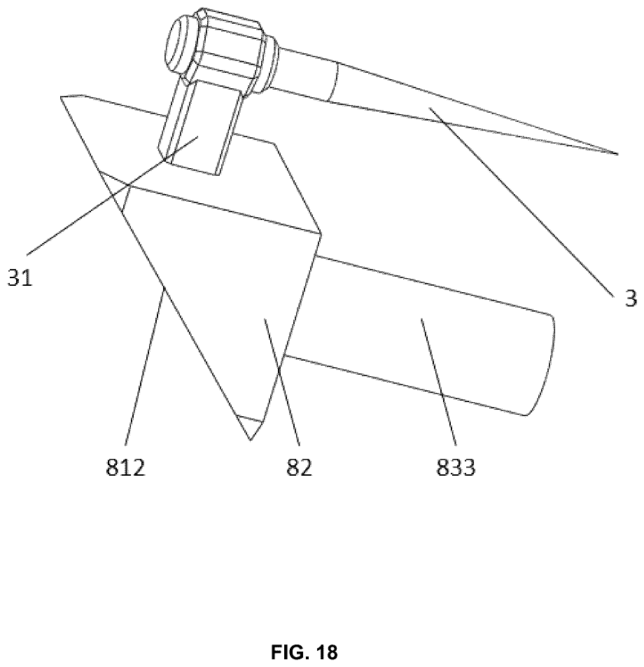

In some embodiments, the piercing base 72 includes a main base 82 (see FIG. 18). In some embodiments, the anastomosing surface 812 is provided on the main base 82 of the piercing base 72, and the needle holder 31 with the piercing needles 3 is provided above the main base 82, referring to FIGS. 13-16.

In some embodiments, when the inhaler is a single-side piercing type, the inhaler includes one slope-type inclined track 81 and one main base 82, and two parallel piercing needles 3 are provided on one needle holder 31, and two piercing channels 12 arranged in parallel are provided on the same side of the inhaler. In some embodiments, when the inhaler is a double-side piercing type, two slope-type inclined tracks 81 are symmetrically provided on the left and right sidewalls of the lower structure 2, and two main bases 82 corresponding to the two slope-type inclined tracks 81 are provided, one needle holder 31 is provided with one piercing needle 3, and two piercing channels 12 arranged in parallel or in the same straight line are provided on both sides of the inhaler, referring to FIGS. 6 and 13.

In some embodiments, the inhaler further includes a fitting structure for fitting the main base 82 on the inclined track. In some embodiments, as shown in FIG. 13, the fitting structure includes a spring structure 831 providing elasticity on the side, one end of the spring structure 831 is attached to a lower end of the upper structure 1, and the other end of the spring structure 831 is attached to a side of the main base 82. The arrangement of the spring structure 831 can maintain the main base 82 to fit on the inclined track by the elasticity to ensure the mating movement of the parts, referring to FIGS. 13 and 14.

In some embodiments, the spring structure 831 is confined within a parallel lumen 832 that is parallel to the piercing channel 12, the parallel lumen 832 includes a lumen attached to the lower portion of the upper structure 1 and a parallel rod 833 attached to a side portion of the main base 82, and the parallel rod 833 always has a socketed segment with the parallel lumen 832. This arrangement ensures that the spring structure 831 provides a stabilizing force, which further maintains the elasticity stability of the spring structure 831. The parallel lumen 832 may protrude in the lower portion of the upper structure 1 or set on the sidewall of the upper structure 1, referring to FIGS. 13 and 14.

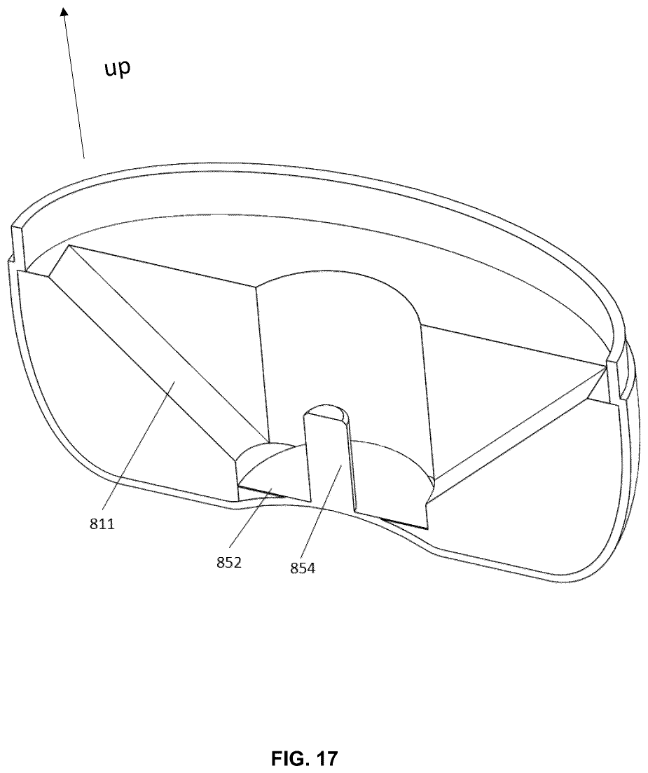

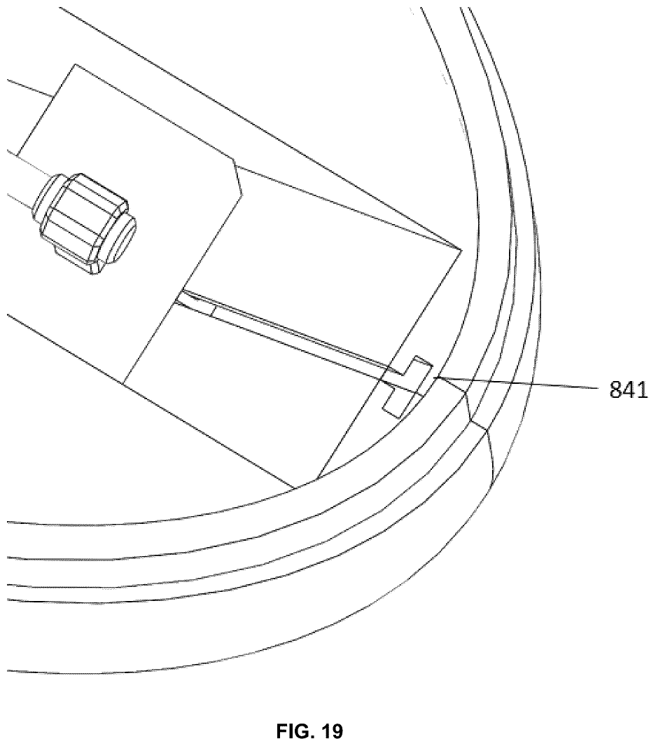

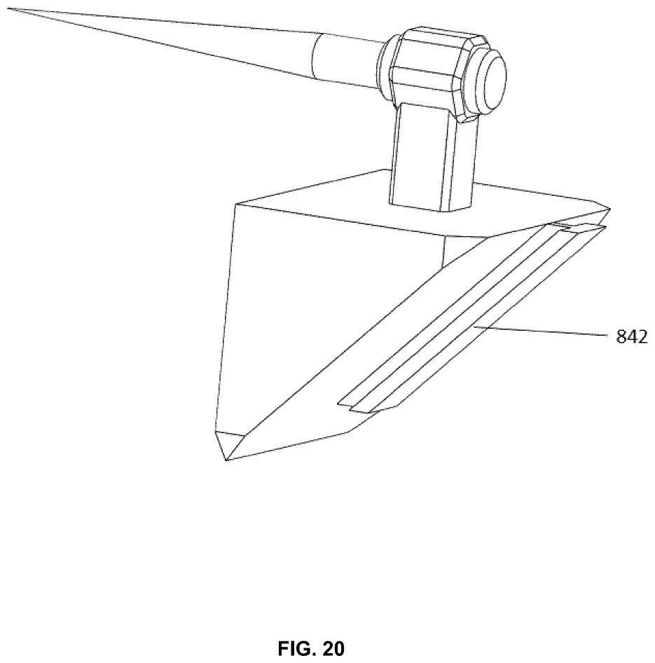

FIG. 17 is a schematic diagram illustrating a left-right longitudinal cross-sectional structure of the lower structure of the slope-type inclined track embodiment according to some embodiments of the present disclosure; FIG. 18 is a schematic diagram illustrating a structure of a piercing base with parallel rods of the slope-type inclined track embodiment according to some embodiments of the present disclosure; FIG. 19 is a schematic diagram illustrating a partially enlarged structure of T-shaped limiting grooves of the slope-type inclined track embodiment according to some embodiments of the present disclosure; and FIG. 20 is a schematic diagram illustrating a piercing base with T-shaped protrusions of the slope-type inclined track embodiment according to some embodiments of the present disclosure.

As shown in FIGS. 17-20, in other embodiments, the fitting structure is a T-shaped limiting groove 841 provided on the inclined surface 811, and the fitting structure further includes a T-shaped protrusion 842 protruding out of the anastomosing surface 812, the T-shaped protrusion 842 being adapted to the T-shaped limiting groove 841, which can achieve that the anastomosing surface 812 is always fitted with the inclined surface 811. In some embodiments, the parallel lumen 832 may also be provided to increase structural stability, the parallel lumen 832 includes a lumen attached to the lower portion of the upper structure 1 and a parallel rod 833 attached to the side of the main base 82, and the parallel lumen 832 and the parallel rod 833 have a socketed segment. Such arrangement ensures that the spring structure 831 provides a stable force. The parallel lumen 832 may protrude in the lower part of the upper structure 1 or provided on the sidewall of the upper structure 1, referring to FIG. 14.

In some embodiments, with reference to FIGS. 10 and 14, a secondary adapter structure may be provided within the parallel lumen 832 and the parallel rod 833, which is correspondingly a secondary adaptation groove 724 and a secondary adaptation protrusion 725. The secondary adapter structure can enhance the adaptation between the parallel lumen 832 and the parallel rod 833, increasing the structural stability.

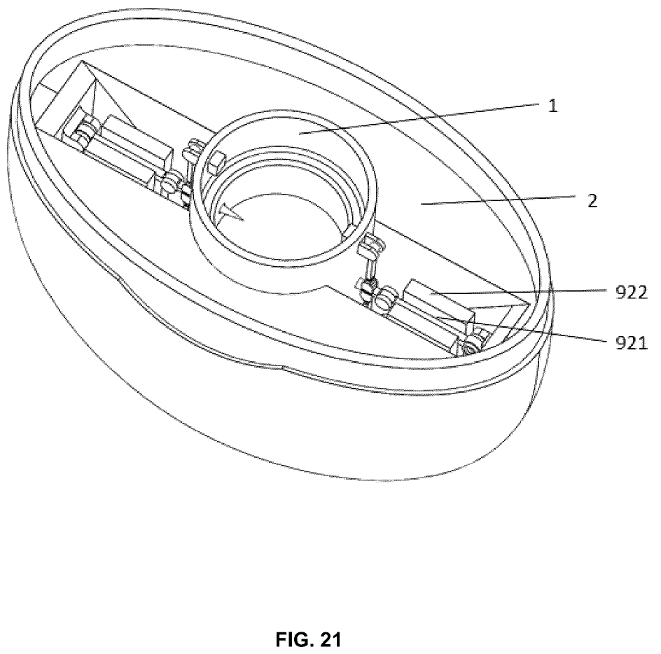

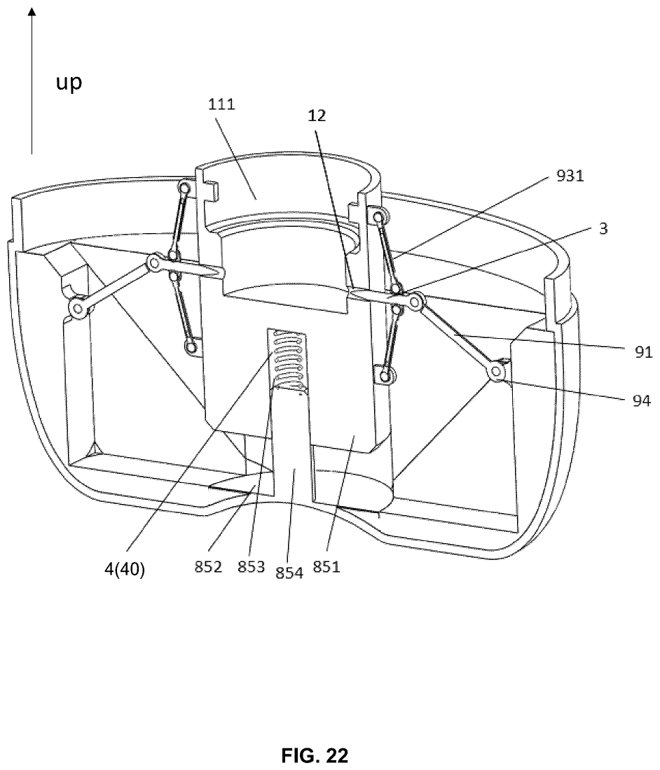

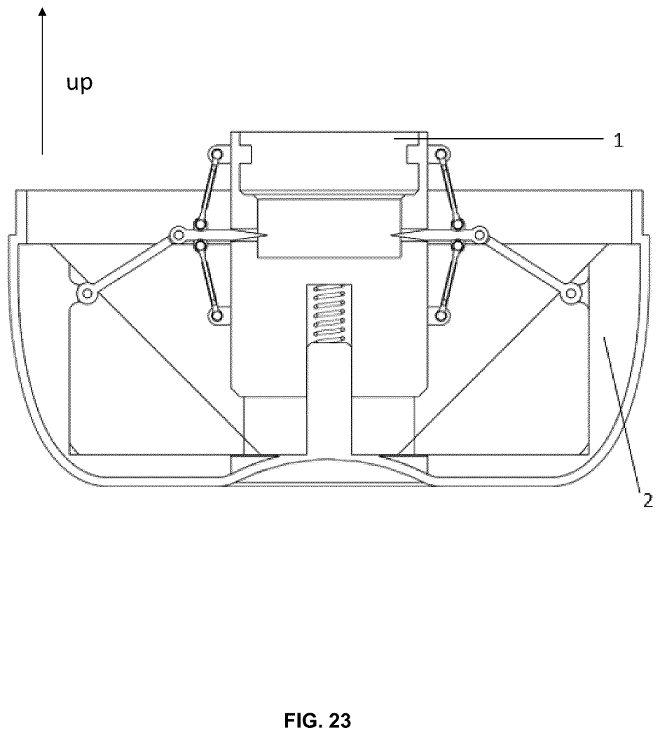

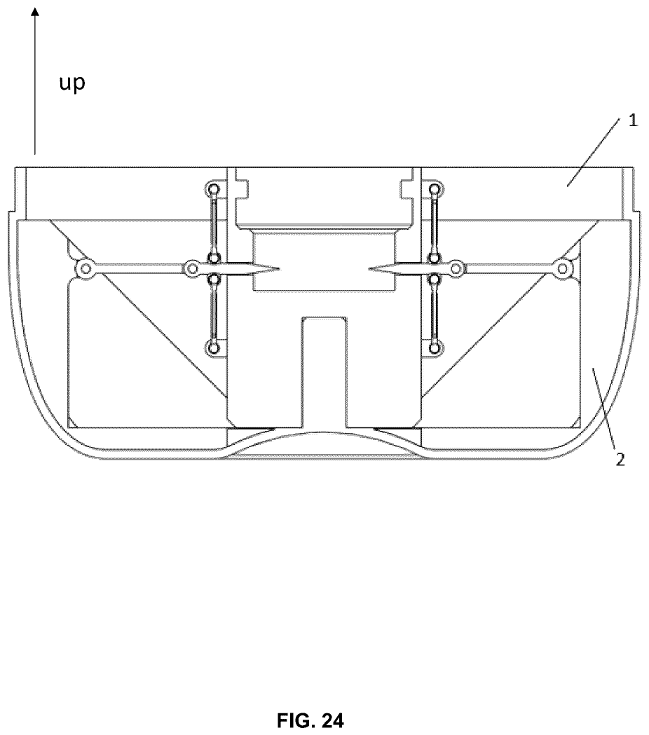

FIG. 21 is a schematic diagram illustrating a structure of the upper structure and the lower structure of a tilting main connecting rod embodiment, with the upper structure not showing the finger placement area according to some embodiments of the present disclosure; FIG. 22 is a schematic diagram illustrating a left-right longitudinal cross-sectional structure of the upper structure and the lower structure of the tilting main connecting rod embodiment, with the upper structure not showing the finger placement area according to some embodiments of the present disclosure; FIG. 23 is a schematic diagram illustrating a left-right longitudinal cross-sectional structure of the tilting main connecting rod embodiment in an unpierced state according to some embodiments of the present disclosure; and FIG. 24 is a schematic diagram illustrating a left-right longitudinal cross-sectional structure of the tilting main connecting rod embodiment in a pierced state according to some embodiments of the present disclosure.

In some embodiments, the drive structure includes a linkage structure connected to the piercing base 72 and a hinge structure. The linkage structure refers to a structure for realizing the piercing movement by rotation of the linkage. The hinge structure is configured to realize the hinge of the linkage structure with the lower structure 2 and the piercing base 72. In some embodiments, the linkage structure includes at least one main connecting rod 91 obliquely connected to the piercing base 72, one end of the main connecting rod 91 is hinged to the sidewall of the lower structure 2 by the hinge structure, and the other end of the main connecting rod 91 is hinged to the piercing base 72 through the hinge structure. When the upper structure 1 and the lower structure 2 undergo the longitudinal linear relative movement, the main connecting rod 91 drives the piercing needles 3 to pierce a capsule in the capsule chamber 111 along the piercing channel 12, referring to FIGS. 21-24.

In some embodiments, the main connecting rod 91 is tilted in a direction gradually tilting upwardly from the position at the upper edge of the lower structure 2 and toward the capsule chamber 111. This way can realize the change of the horizontal distance of the main connecting rod 91 during the upward movement of the lower structure 2 and realize the piercing movement of the piercing base 72 toward the capsule chamber 111 when the lower structure 2 moves relatively upward, see FIG. 22.

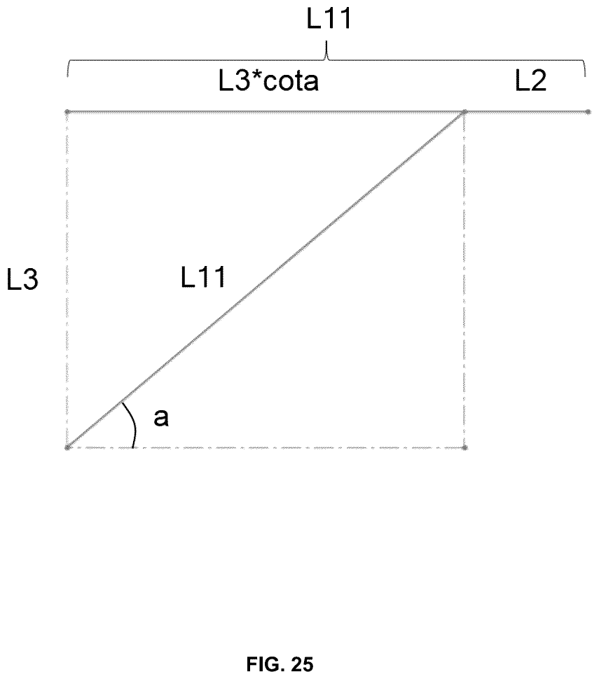

FIG. 25 is a schematic diagram illustrating a relationship between an incline angle of the main connecting rod, a length of the main connecting rod, a piercing distance of the piercing needles, and a longitudinal linear movement distance between the upper structure and the lower structure according to some embodiments of the present disclosure.

In some embodiments, the direction of the piercing channel 12 is the horizontal direction or an inclined upward or inclined downward direction. Taking the direction of the piercing channel 12 being a horizontal direction as an example, a relationship between the incline angle a of the main connecting rod 91, the length L11 of the main connecting rod 91, the piercing distance L2, and the longitudinal linear movement distance L3 of upper structure 1 and the lower structure 2 is represented as L11≥L2+L3*cota, referring to FIG. 25. This setting can determine the remaining indicators after determining any three indicators according to the need. In some embodiments, the piercing base 72 includes one or more piercing needles 3 provided with a connecting portion of connecting rod 94 or a needle holder 31 with the piercing needles 3. Exemplarily, the piercing base 72 is the piercing needles 3 provided with the connecting portion of connecting rod 94. The piercing needles 3 are contained in a portion of the piercing channel 12 at any time, which ensures that the piercing needles 3 move along the piercing channel 12, referring to FIG. 22.

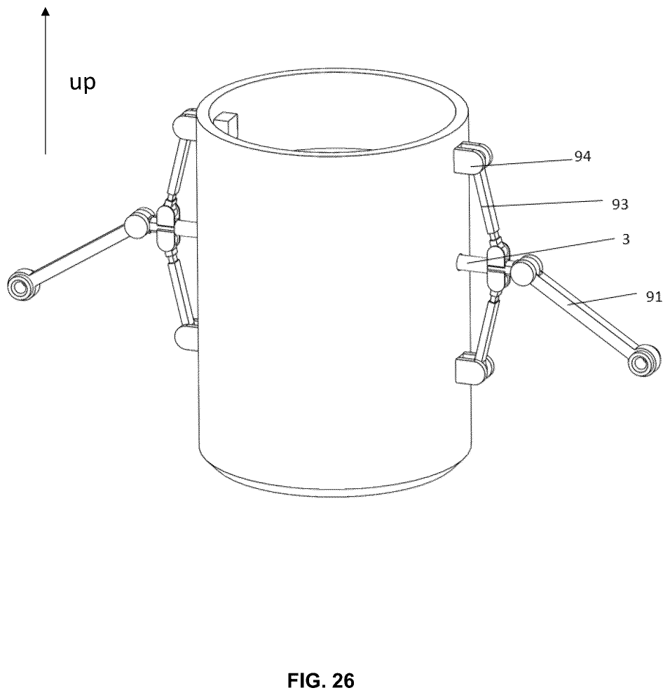

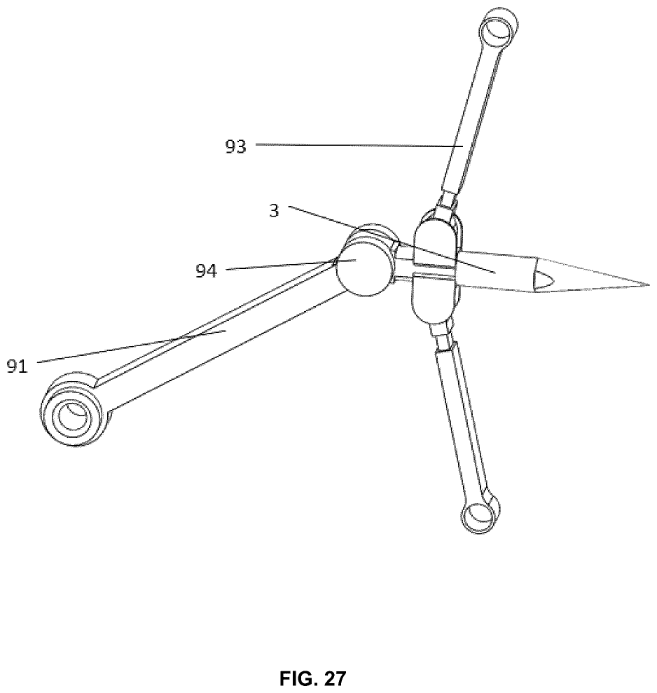

FIG. 26 is a schematic diagram illustrating a structure of the upper structure, a linkage structure, and piercing needles in a combined state of the tilting main connecting rod embodiment according to some embodiments of the present disclosure; and FIG. 27 is a schematic diagram illustrating a structure of the linkage structure and the piercing needles in a combined state of the tilting main connecting rod embodiment according to some embodiments of the present disclosure.

In some embodiments, the inhaler further includes a direction maintaining structure for maintaining that the main connecting rod 91 moves in a plane. In some embodiments, the direction maintaining structure is a running channel 921 that has the same thickness or diameter as the main connecting rod 91, the running channel 921 being formed by two plate structures 922 extending out on the side of the lower structure 2. In some embodiments, the direction maintaining structure may also be two maintaining connecting rods 93 connected to the upper structure 1, the two maintaining connecting rods 93 are provided on both sides of the piercing channel 12, one end of each of the maintaining connecting rods 93 is pivotally connected (e.g., hinged) to the sidewall of the upper structure 1, the other end of each of the maintaining connecting rods 93 is movably connected to the piercing needles 3 or the needle holder 31, and the two maintaining connecting rods 93 are symmetrically provided around the piercing channel 12. In some embodiments, the arrangement of the maintaining connecting rod 93 and the main connecting rod 91 also serve as an anti-separation structure, and the anti-separation structure refers to a structure capable of preventing the separation of the upper structure 1 from the lower structure 2, see FIGS. 21, 26, and 27. For more description of the anti-separation structure, see the related descriptions later.

In some embodiments, the bottom of the upper structure 1 and the bottom surface of the lower structure 2 are provided with mutually socketed lumen structures in correspondence, and the lumen structures include an upper lumen 41 at the bottom of the upper structure 1 and a lower lumen 42 extending from the bottom surface of the upper structure 1, the upper lumen 41 is socketed on the outer side of the lower lumen 42 or the upper lumen 41 extends into the inner side of the lower lumen 42, and the reset spring 40 is provided in the center of the upper lumen 41 and the lower lumen 42, referring to FIGS. 6 and 7.

In some embodiments, as shown in FIGS. 10 and 12, the inhaler further includes a direction-guiding structure for maintaining a longitudinal linear movement between the upper structure 1 and the lower structure 2, the direction-guiding structure includes a direction-guiding groove or a direction-guiding column 572 arranged in the middle of the upper structure 1, and the direction-guiding structure further includes a direction-guiding column or a direction-guiding groove 571 arranged in the middle of the lower structure 2 in correspondence, the direction-guiding groove and the direction-guiding column arranged in different structures are adapted to each other, and the direction of the direction-guiding groove and the direction-guiding column (i.e., the length direction) is a longitudinal straight line.

In some embodiments, the lower lumen 42 is socketed inside the upper lumen 41, and the direction-guiding column 572 is provided on the outside of the outer sidewall of the upper lumen 41, referring to FIGS. 7, 10, and 12.

As shown in FIGS. 7 and 13, in other embodiments, a bottom cavity 852 is provided within the lower structure 2 for the movement of the bottom structure 851 of the upper structure 1, and the bottom structure 851 is adapted to the bottom cavity 852 in shape, a reset spring 40 is provided within the bottom cavity 852, and a portion of the bottom structure 851 is always provided within the bottom cavity 852, which can also well realize guidance of the longitudinal linear movement.

In some embodiments, as shown in FIG. 14, the bottom structure 851 may be provided with a secondary adaptation cavity 853, a secondary adaptation rod 854 extends from the bottom cavity 852, and the secondary adaptation cavity 853 adapts to the secondary adaptation rod 854. The secondary adaptation rod 854 extends into the secondary adaptation cavity 853, and the reset spring 40 is provided in the secondary adaptation cavity 853. This arrangement can further strengthen the guidance of the movement direction and adaptation effect, ensuring the stability of movement.

In some embodiments, the inhaler may also include an anti-separation structure, which can both ensure longitudinal linear movement and prevent separation of the upper structure 1 from the lower structure 2 after the upper structure 1 is combined with the lower structure 2. The anti-separation structure may be implemented in any locking manner. In some embodiments, as shown in FIGS. 2 and 12, the anti-separation structure includes a limit rod 51 extending from below the upper structure 1, a locking protrusion 52 is provided at the lowermost part of the limit rod 51, the length of the limit rod 51 is not less than the longitudinal linear movement distance between the upper structure 1 and the lower structure 2, and the lower structure 2 is provided with a limit protrusion 54 adapted to the locking protrusion 52, and a position of the locking protrusion 52 contacting the limit protrusion 54 is an anti-separation position, and other positions are positions for maintaining the movement state. The other positions refer to positions other than the anti-separation position in which the locking protrusion 52 is located during the process of the user pressing the inhaler.

In some embodiments, a length of the limit rod 51 from the locking protrusion 52 to the lowermost end of the upper structure 1 is equal to the longitudinal linear movement distance between the upper structure 1 and the lower structure 2.

In some embodiments, the limit protrusion 54 is a protruding structure protruding from the sidewall of the lower structure 2.

Exemplarily, a movement groove 53 for movement of the locking protrusion 52 is correspondingly provided on the sidewall of the lower structure 2, and a limit protrusion 54 adapted to the locking protrusion 52 is provided on the uppermost part of the movement groove 53, and the limit protrusion 54 is a top body from the bottom of the movement groove to the sidewall of the lower structure 2 provided at the top of the movement groove 53, as shown in FIGS. 2 and 11, and the length of the movement groove 53 is equal to the length of the limit rod 51, as shown in FIGS. 2 and 12.

In some embodiments, a guide groove 55 is provided above the movement groove 53, a direction and a width of the guide groove 55 are identical to a direction and a width of the movement groove 53, a lowermost end of the guide groove 55 is an uppermost part of the limit protrusion 54, a guide surface 56 is provided at the uppermost part of the limit protrusion 52, which helps to make the limit rod 51 easier to enter. In some embodiments, at least one pair of limit rods 51 and corresponding movement grooves 53 are provided symmetrically on the left and right sidewalls or on front and rear sidewalls to ensure stable and balanced arrangement. In some embodiments, two pairs of limit rods 51 and corresponding movement grooves 53 may be symmetrically provided around the capsule chamber 111 on the left and right sidewalls or on the front and rear sidewalls, referring to FIGS. 2 and 12.

As shown in FIG. 22, in other embodiments, the anti-separation structure includes a connecting structure interconnecting without interfering with the longitudinal linear movement. In some embodiments, the connecting structure is an interconnecting link, and the interconnecting link is provided with connecting portions at the connection with both the upper structure 1 and the lower structure 2. For example, the interconnecting link may be four interconnected diamond-shaped links, both side ends of which are free, and the upper and lower sides of which are respectively connected with the upper structure 1 and the lower structure 2. For another example, as shown in FIG. 21, the interconnecting link includes at least one main connecting rod 91 connected to the side edge of the lower structure 2, the other end of the main connecting rod 91 is connected to the piercing structure, and the piercing structure always includes a portion of piercing needles within a piercing channel. The piercing structure refers to a structure composed of the piercing needles 3, the needle holder 31, and the piercing base 72, the piercing channel may refer to the piercing channel 12, and the piercing needles may refer to the piercing needles 3. In this way, it is possible to realize not only the anti-separation, but also the piercing movement of the piercing needles by the longitudinal linear relative movement through the main connecting rod 91. The main connecting rod 91 moves in a plane, referring to FIGS. 22-24.

In some embodiments, the inhaler may also be provided with a structure that facilitates operation, as shown in FIG. 7. In some embodiments, in a stationary state, an anastomosis table 612 shaped in line with the upper cavity of the lower structure 2 is provided above the upper structure 1, an upper plane of the anastomosis table 612 is parallel to an upper plane of the upper cavity of the lower structure 2, the anastomosis table 612 is perpendicularly in and out of the upper cavity, and an operation table 61 is provided above the anastomosis table 612, and finger placement structures 613 are provided on both sides of the operation table 61.

In some embodiments, a positional relationship between the anastomosis table 612 and the operation table 61 is set as the distance between the anastomosis table 612 and the operation table 61 being not less than the longitudinal linear movement distance between the upper structure 1 and the lower structure 2, which ensures an easy operation and prevents the operation table 61 from entering the upper cavity of the lower structure 2, which is not good for operation. In some embodiments, the operation table 61 has the same peripheral shape as the anastomosis table 612.

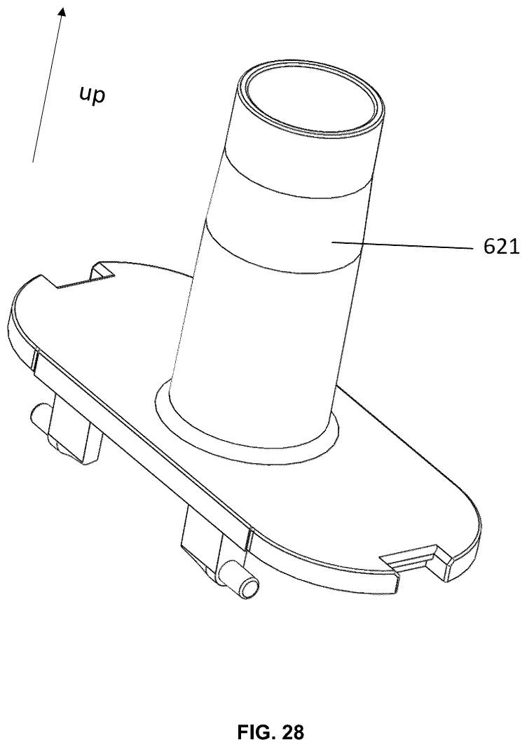

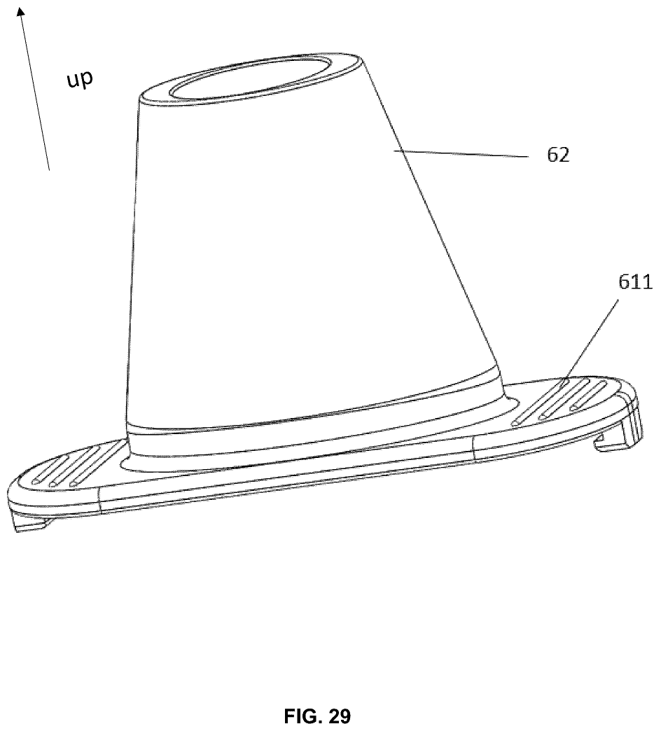

FIG. 28 is a schematic diagram illustrating a structure of an inhalation channel section according to some embodiments of the present disclosure; and FIG. 29 is a schematic diagram illustrating a structure of the nozzle section according to some embodiments of the present disclosure.

In some embodiments, an inhalation channel section 62 with a drug inhalation channel is also provided on the upper structure 1, a nozzle section 63 with an anastomosis suction nozzle is also sleeved on the inhalation channel section 62, the bottom of the inhalation channel section 62 and the bottom of the nozzle section 63 are provided with peripheral arc structures consistent with the shape of the anastomosis table 612, and the bottom of the uppermost nozzle section 63 is the operation table 61. This arrangement ensures that the combined structure is neat and stable and the hand placement operation is accomplished through the arc structure on the nozzle section 63. An anti-slip pattern 611 is provided on the arc structure of the operation table 61 to increase the friction and controllability during operation, referring to FIGS. 6, 7, and 29.

Exemplarily, the combination of the inhalation channel section 62 with the other embodiments of the present disclosure is described as follows. The inhalation channel section 62 with the drug inhalation channel is also provided on the upper structure 1, and the inhalation channel is provided on the upper structure 1 through a rotational combination or a snap-on combination. The inhalation channel section 62 is provided with a columnar channel tube 621. A barrier mesh is provided at the bottom of the inhalation channel section 62, and the barrier mesh is configured to prevent the capsule from being inhaled during inhalation of the dry powder. The nozzle section 63 with an anastomosis suction nozzle is also sleeved over the inhalation channel section 62, and an anastomosis tube 631 that is consistent with the columnar channel tube 621 is internally arranged above the nozzle section 63. This setting increases the adaptation degree between the nozzle section 63 and the inhalation channel section 62. The bottom of the inhalation channel section 62 and the bottom of the nozzle section 63 are provided with arc structures that are consistent with a shape of the operation table 61, which ensures that the combined structure is neat and stable. The hand placement operation is accomplished through the arc structure on the nozzle section 63, and the anti-slip pattern 611 is provided on the arc structure on the nozzle section 63 to increase the friction and controllability in the operation process. A vertically snapping protective cap 64 or a rotationally opening and closing protective cap 64 is also provided above the nozzle section 63 to prevent contamination, referring to FIGS. 1, 7, 28, and 29. By placing the index finger and the middle finger at the finger placement structure 613 and placing the thumb at a bottom of the lower structure 2, it can complete the relative movement between the upper structure 1 and the lower structure 2 through cooperation of the three fingers, thereby realizing the purpose of piercing the capsule from the side by the longitudinal linear movement.

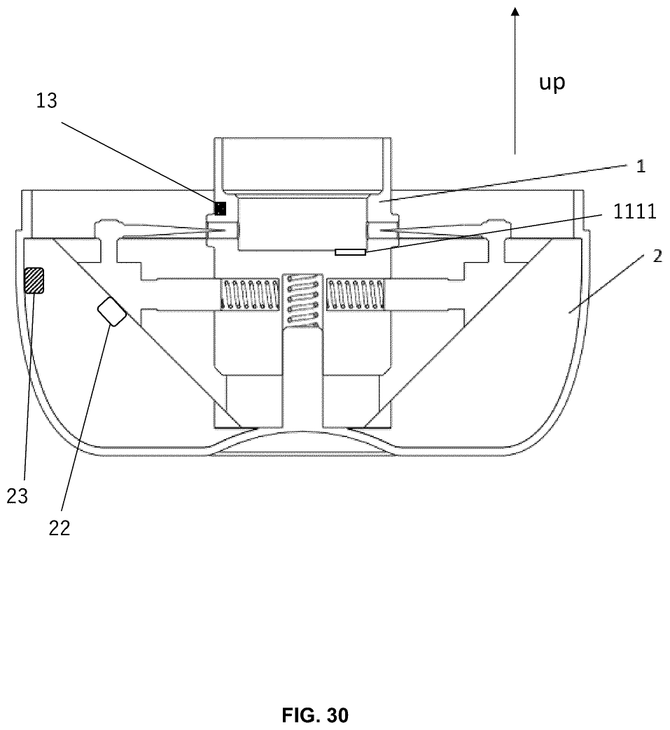

FIG. 30 is a schematic diagram illustrating a left-right longitudinal cross-sectional structure of the slope-type inclined track embodiment in an alternative pierced state according to some embodiments of the present disclosure.

In some embodiments, referring to FIG. 30, the bottom of the capsule chamber 111 is also provided with a weight sensor 1111, a controller 13 is also provided within the upper structure 1, and the controller 13 includes a Bluetooth module (not shown in the FIG. 30). The weight sensor 1111 is configured to monitor a weight within the capsule chamber 111 and send a weight monitoring signal to the controller 13 at a preset time interval.

The weight sensor 1111 refers to a high-precision weight sensor (e.g., a sensor with 1 mg resolution), including a strain gauge weighing sensor, a Micro-Electro-Mechanical System (MEMS) sensor, etc., which is configured to monitor the weight within the capsule chamber 111.

The preset time interval refers to a predetermined time interval at which the weight sensor 1111 sends a weight monitoring signal. In some embodiments, the preset time interval may be set by manual experience, e.g., 2 seconds.

The weight monitoring signal refers to an electrical signal reflecting weight information within the capsule chamber 111. The weight monitoring signal is generated by the weight sensor 1111 based on the monitored weight within the capsule chamber 111.

The controller 13 refers to a device for receiving sensor signals and information communication, for example, a Programmable Logic Controller (PLC) chip, etc.

The Bluetooth module refers to a module that enables communication connection between the controller 13 and other devices (e.g., a user terminal device, the weight sensor, etc.).

The user terminal device refers to a device that provides a user with functions such as information display, prompts, data interaction, or the like. In some embodiments, the user terminal device may include a cell phone, a tablet, a computer, or the like.

In some embodiments, the weight sensor 1111 monitors the weight within the capsule chamber 111 in real time and sends the weight monitoring signal to the controller 13 according to a preset time interval via a wired connection or other communication means (e.g., Bluetooth, etc.).

In some embodiments of the present disclosure, by setting a weight sensor to monitor the weight in the capsule chamber, it can be convenient for the user to know the medication usage.

In some embodiments, the controller 13 is configured to: in response to determining that the weight detected by the weight monitoring signal does not change for N consecutive time points, send a monitoring signal sequence from a first time point to a current time point to the user terminal device. In some embodiments, the user terminal device determines, based on the monitoring signal sequence, an abnormality state category by an abnormality prediction model, and performs an information prompt based on the abnormality state category; and the abnormality prediction model is a machine learning model.

In some embodiments, a value of N among the N time points may be set by manual experience. N may be a positive integer, e.g., N=10.

The monitoring signal sequence refers to a vector sequence constructed based on the weight monitoring signals from a first time point to a current time point.

The first time point refers to a time point at which the placed capsule contacts the bottom of the capsule chamber 111.

The abnormality state category refers to a label reflecting a category of possible anomalies of the capsule chamber 111 during the operation, for example, the abnormality state category may include failure to pierce or inhale after placing the drug, completion of drug inhalation, drug deterioration, or the like.

In some embodiments, the user terminal device determines the abnormality state category based on the monitoring signal sequence through the abnormality prediction model.

In some embodiments, the abnormality prediction model may be a machine learning model. For example, the abnormality prediction model may be a Neural Networks (NN) model, a Deep Neural Networks (DNN) model, etc. or any combination thereof. In some embodiments, an input of the abnormality prediction model includes a monitoring signal sequence, and an output of the abnormality prediction model includes an abnormality state category.

In some embodiments, the abnormality prediction model may be obtained by training a training data set. For example, the training includes obtaining the training data set and performing multiple rounds of iterations, and the at least one round of iterations includes: selecting one or more training samples from the training data set, inputting the one or more training samples into an initial abnormality prediction model, obtaining model output corresponding to the one or more training samples; substituting the model output corresponding to the one or more training samples and labels of the one or more training samples into a predefined loss function, and calculating a value of the loss function; and updating parameters of the initial abnormality prediction model based on the value of the loss function by gradient descent or other methods. When an end-of-iteration condition is satisfied, the iteration is ended, and a trained abnormality prediction model is obtained. The end-of-iteration condition may be that the loss function converges, a count of the rounds of iterations reaches a threshold, etc.

In some embodiments, the training samples may include at least a historical monitoring signal sequence. The training labels may characterize the abnormality state categories corresponding to the training samples. The training data set includes a plurality of training samples and training labels corresponding to the plurality of training samples. In some embodiments, an abnormality state category and an abnormality occurrence time point when an abnormality occurs may be obtained by counting the historical weight monitoring signals, the abnormality state category may be used as the training label, and the monitoring signal sequence from a historical first time point to the abnormality occurrence time point may be used as the training sample corresponding to the training label. Exemplarily only, for a training sample, if the historical monitoring signal sequence from the historical first time point to the abnormality occurrence time point indicates a small change in weight and a change trend from fast to slow, then the drug deterioration is used as a corresponding training label; if it indicates that the weight change is large and nearly linear, the completion of drug inhalation is used as a corresponding training label; and if it indicates that the weight remains unchanged, the failure to pierce or inhale after placing the drug is used as a corresponding training label.

The abnormality occurrence time point is a moment when an abnormality state occurs within the capsule chamber 111. For example, a first time point when the weight is constant in the historical monitoring signal sequence may be used as the abnormal occurrence time point.

In some embodiments, the user terminal device performs an information prompt based on the abnormality state category.

In some embodiments, after determining the abnormality state category, the user terminal device may send the information prompt to the user in a variety of ways (e.g., text prompt, sound reminder, vibration, etc.). Taking the text prompt as an example, if the abnormality state category is the failure to pierce or inhale after placing drug, a prompt box reading “Please press the device to pierce the capsule” pops up on the display screen of the user terminal device; if the abnormality state category is the completion of drug inhalation, a prompt box reading “The drug inhalation has been completed, please take out the capsule” pops up on the display screen of the user terminal device; and if the abnormality state category is the drug deterioration, a prompt box reading “Drug deterioration, no inhalation, please take out the capsule immediately” pops up on the display screen of the user terminal device.

In some embodiments of the present disclosure, by utilizing the abnormality prediction model obtained by training, the abnormality state category can be automatically determined and prompted on the user terminal device, so as to avoid drug waste or inhalation of deteriorated drug resulted from empirical estimation, thereby improving the accuracy of drug usage.

In some embodiments, in response to the abnormality state category being failure to pierce or inhale after placing the drug, the user terminal device sends the abnormality state category to the controller 13; the controller 13 obtains a user pressing pressure in the subsequent time period, and in response to the user pressing pressure meeting a preset condition, sends a piercing needle damage warning to the user terminal device, and after receiving the piercing needle damage warning, the user terminal device displays a prompt for checking the piercing needles.

The subsequent time period refers to a period of time after the controller 13 receives the abnormality state category transmitted by the user terminal device. In some embodiments, a duration of the subsequent time period may be pre-set by manual experience, for example, the subsequent time period is 10 seconds.

The user pressing pressure refers to a pressure at which the user presses the inhaler. In some embodiments, the user pressing pressure may be obtained by monitoring via, for example, the pressure sensor 22.

The preset condition refers to a condition used to determine whether the piercing needles 3 are damaged. In some embodiments, the preset condition may be that the user pressing pressure is within an optimal pressure range and the weight monitoring signal is not changed for M consecutive time points after the pressing.

The optimal pressure range refers to a pressure range that is capable of piercing the capsule and not damaging the inhaler. In some embodiments, the optimal pressure range may be preset by the skilled person based on experience, e.g., 1.2 N to 1.8 N.

In some embodiments, a value of M among the M consecutive time points may be set by manual experience. M is a positive integer, e.g., M=5.

The piercing needle damage warning refers to a warning signal reflecting that the piercing needles 3 are likely to be damaged.

The prompt for checking the piercing needles refers to a prompt message used to remind the user to check the state of the piercing needles 3. In some embodiments, the prompt for checking the piercing needles may remind the user in a variety of ways, such as text, sound, vibration.

In some embodiments, in response to determining that the user pressing pressure satisfies the preset condition, the controller 13 sends the piercing needle damage warning to the user terminal device, and the user terminal device displays the prompt for checking the piercing needles after receiving the piercing needle damage warning.

In some embodiments, the controller 13 may send the piercing needle damage warning to the user terminal device via a Bluetooth module.

Exemplarily, after the user terminal device receives the piercing needle damage warning, a prompt box reading “Please check whether the piercing needles are damaged” may pop up on the display screen of the user terminal device.

In some embodiments of the present disclosure, by combining the weight monitoring and the pressure monitoring, the possible reasons for the occurrence of the abnormal state are analyzed (e.g., the weight in the capsule chamber 111 remains unchanged because the user does not press it, the piercing needles 3 cannot pierce the capsule because it is damaged, etc.), which helps to determine malfunctioning situation in time and guide the user to operate correctly, so as to enhance the user experience.

In some embodiments, the inclined track is provided with a pressure sensor 22 and a vibration device 23. In some embodiments, both the pressure sensor 22 and the vibration device 23 are communicatively connected to the controller 13.

The pressure sensor 22 refers to a sensor that is used to monitor the user pressing pressure.

The vibration device 23 refers to a device for prompting the user through vibration. For example, the vibration device 23 may include a miniature eccentric rotary motor, a piezoelectric ceramic vibrator (PZT), or the like.

In some embodiments, the controller 13 is configured to: in response to determining that the user pressing pressure monitored by the pressure sensor 22 is greater than an optimal pressure range, control the vibration device 23 to vibrate at a high frequency; and in response to determining that the user pressing pressure monitored by the pressure sensor 22 is less than the optimal pressure range, control the vibration device 23 to vibrate at a low frequency. It should be understood that when the user pressing pressure monitored by the pressure sensor 22 is within the optimal pressure range, the user may normally press the inhaler for drug and does not damage the inhaler, and there is no need for prompting by the vibration device 23 at this time.

When the user presses the inhaler, excessive pressure may damage the device, and insufficient pressure may fail to completely pierce the capsule. In some embodiments of the present disclosure, by setting the pressure sensor 22 and the vibration device 23, the user can be reminded and instructed to adjust the applied pressure using the vibrations at different frequencies from the vibration device 23, so as to quickly pierce the capsule while avoiding damage to the inhaler.

In some embodiments, the user terminal device may adjust the optimal pressure range based on a capsule type, a count of times of using the piercing needles, and a historical pressing record.

The capsule type refers to a type to which different capsules belong. For example, the capsule type may include a single-layer capsule, a multi-layer capsule, a microcapsule, or the like. In some embodiments, the user may select the capsule type on the user terminal device when placing the capsule into the capsule chamber 111. In some embodiments, different optimal pressure ranges corresponding to different capsule types may be stored on the user terminal device. Exemplarily, each capsule type may correspond to a set of initially preset baseline lower limit and baseline upper limit, and a range between the baseline lower limit and the baseline upper limit is the optimal pressure range for the corresponding capsule type.

The count of times of using the piercing needles refers to a total count of times of the piercing needles 3 being pressed in the inhaler. In some embodiments, the count of times of using the piercing needles may be obtained based on the count of times of the pressure sensor 22 monitoring that the pressure of the piercing needles 3 is greater than 0. The count of times of using the piercing needles is 0 if there is no relevant record.

The historical pressing record refers to a record of the user pressing pressure in the historical record. In some embodiments, the historical pressing record may be obtained based on the maximum pressure monitored by the pressure sensor 22 during each pressing by the user. The historical pressing record is recorded as 0 if there is no relevant record.

In some embodiments, the user terminal device may adjust the optimal pressure range based on the capsule type, the count of times of using the piercing needles, and the historical pressing record in a plurality of ways. Exemplarily, the user terminal device may adjust the optimal pressure range by operations as follows.
1) The capsule type entered by the user is obtained, and the baseline upper limit and baseline lower limit corresponding to the capsule type is further obtained.
2) Based on the baseline upper limit and baseline lower limit corresponding to the capsule type, the count of times of using the piercing needles, and the historical pressing record, the adjusted optimal pressure range is determined by the optimal lower limit formula and the optimal upper limit formula.

The optimal lower limit formula refers to a formula for determining a lower limit of the adjusted optimal pressure range. Exemplarily, the optimal lower limit formula may be expressed as formula (1) below:

$${P}_{d}^{′} = {P}_{d} + n / 100 + b . \qquad ( 1 )$$

Where P_{d}′ denotes the lower limit of the adjusted optimal pressure range, P_{d} denotes the baseline lower limit, n denotes the count of times of using the piercing needles, and b denotes an offset (see description below for details).

The optimal upper limit formula refers to a formula for determining the upper limit of the adjusted optimum pressure range. Exemplarily, the optimal upper limit formula may be expressed as formula (2) below:

$${P}_{u}^{′} = {P}_{u} + n / 100 + b . \qquad ( 2 )$$

Where Pᵤ′ denotes the upper limit of the adjusted optimal pressure range, Pᵤ denotes the baseline upper limit, n denotes the count of times of using the piercing needles, and b denotes the offset (see description below for details). Parameters in the above formulas (1) and (2) are dimensionless values.

After the above calculations to obtain P_{d}′ and Pᵤ′, the adjusted optimal pressure range is P_{d}′N˜Pᵤ′N.

Offset is an adjustment amount associated with the historical pressing record. In some embodiments, the controller 13 may determine the offset based on a preset rule based on the count of times of using the piercing needles and the historical pressing record. For example, the preset rule may be as follows: if the count of times of using the piercing needles is 0, the offset is set to 0; if the count of times of using the piercing needles is not 0 and an average of the historical pressing records is greater than a preset pressure, then the offset is set to +0.2; if the count of times of using the piercing needles is not 0 and the average of the historical pressing record is less than or equal to the preset pressure, the offset is set to −0.2. The preset pressure may be preset by the technician.

3) The user terminal device sends the adjusted optimal pressure range to the controller 13 of the inhaler, and the controller 13 controls the vibration device 23 to vibrate based on the adjusted optimal pressure range. For more details regarding the controller 13 controlling the vibration of the vibration device 23, please see the related descriptions above.

In some embodiments of the present disclosure, by dynamically adjusting the optimal pressure range based on the type of capsule, the count of times of using the piercing needles, and the historical pressing record, it can prevent problems such as mismatched optimal pressure ranges caused by inconsistent piercing force requirements for different capsule types, or the reduced piercing effect due to excessive use of the piercing needles, or different pressing force preferences of different users.

In some embodiments, as shown in FIG. 2, the airflow channel opening 1121 is further provided with a flow velocity sensor 1122. The controller 13 is further configured to: obtain inhalation data based on the flow velocity sensor 1122; determine a user inhalation state and a device leakage state based on the inhalation data; and perform a warning prompt based on the user inhalation state and the device leakage state.

The flow velocity sensor 1122 refers to a sensor configured to monitor a flow velocity at the airflow channel opening 1121. For example, the flow velocity sensor 1122 may be a miniature thermal anemometer, an ultrasonic flow sensor, etc.

The inhalation data refers to relevant flow velocity data reflecting that the user inhales the drug. For example, the inhalation data may be a flow velocity curve monitored by the flow velocity sensor 1122, including flow velocities at different time points.

In some embodiments, the controller 13 may determine the user inhalation state based on the inhalation data using various manners. For example, the controller 13 may determine the user inhalation state based on a relationship between the flow velocity within a first time period in the inhalation data and a preset flow velocity range. The first time period refers to a time period of a first preset duration after the user starts continuous inhalation (e.g., the first preset duration may be set to 3 seconds). For example, if the flow velocity within the first time period remains within the preset velocity range, the user inhalation state is determined to be normal; if the flow velocity within the first time period exceeds the upper limit of the preset velocity range, the user inhalation state is determined to be excessive inhalation; if the flow velocity within the first time period is below the lower limit of the preset velocity range, the user inhalation state is determined to be insufficient inhalation. The preset velocity range may be set by technical personnel.

In some embodiments, the controller 13 may determine the device leakage state based on the inhalation data using various manners. For example, the controller 13 may determine the device leakage state based on the flow velocity within the second time period in the inhalation data. The second time period refers to a time period of a second preset duration when the inhalation process enters a final stage, i.e., after the flow velocity decreases to a preset standard (e.g., the second preset duration may be set to 2 seconds). For example, if the flow velocity within the second time period rapidly drops to zero (e.g., ≤0.1 m/s) and there is no rebound within 2 seconds, it is considered as a normal end of inhalation, and the device leakage state is determined to be no leakage; if the flow velocity within the second time period remains above 0 (e.g., >0.1 m/s) and there is no rebound within 2 seconds, or irregular fluctuations occur, the device leakage state is determined to be presence of leakage.

The warning prompt refers to a prompt for giving a relevant warning to the user. In some embodiments, the controller 13 may perform the warning prompt in various ways based on the inhalation status and the device leakage state. For example, if the user inhalation state is excessive inhalation, a prompt box reading “Current inhalation is excessive” pops up on the display screen of the user terminal device; if the user inhalation state is insufficient inhalation, a prompt box reading “Insufficient inhalation” pops up on the display screen of the user terminal device; and if the device leakage state indicates presence of leakage, a prompt box reading “The device is likely to leak; please check” pops up on the display screen of the user terminal device. In some embodiments, the warning prompt may also be a prompt via sound, vibration, or other means, which are not further described herein.

In some embodiments of the present disclosure, by setting the flow velocity sensor to monitor the user inhalation process, the user inhalation state can be accurately monitored, it can determine whether the inhaler is leaking in time, and the effectiveness of drug delivery and usage safety of the inhaler is ensured by using the warning prompt.

Beneficial effects of the embodiments of the present disclosure include, but are not limited to: for the arrangement of the drive structure, the arrangement of the piercing base and the inclined track or the arrangement of the linkage structure enables that the piercing needles pierce the capsule from the side through the combination with the longitudinal linear movement of the upper structure and the lower structure. The design concept of the inhaler provided in the embodiments of the present disclosure is a new concept for accomplishing the capsule piercing action of the dry powder inhaler by a new form of movement. By such an arrangement, the single-handed horizontal force mode is converted to the single-handed longitudinal force mode, and the longitudinal force mode is easier to operate than the horizontal force mode. Moreover, the aesthetics of the inhaler is higher because the structures are set up in the upper structure and the lower structure. In addition, the overall transverse width of the inhaler can be reduced by converting the direction of the force and it is more convenient to hold the inhaler.

The arrangement of the groove-type inclined tracks on the front and rear sidewalls of the lower structure and symmetrical arrangement of the inclined tracks around the capsule chamber can symmetrically pierce the capsule from both sides, which is safe and effective during dry powder inhalation.

The arrangement of the slope-type inclined track on the left and right sidewalls of the lower structure is also a good technical solution that can realize the corresponding function similar to that of the groove-type inclined track, with remarkable technical effect.

The arrangement of the linkage structure connected to the upper structure in the lower structure can also well realize the effect of piercing the capsule in the capsule chamber along the piercing channel under the premise of guaranteeing that the main connecting rod moves in a motion plane and the idea of arranging specific structures.

The description of the above embodiments is only for understanding the present disclosure. It should be noted that, for a person of ordinary skill in the art, there are a number of improvements that can be made to the present disclosure without departing from the principles of the present disclosure, which will also fall within the protection scope of the claims of the present disclosure.

For each of the patents, patent applications, patent application publications, and other materials cited in the present disclosure, such as articles, books, specifications, publications, documents, or the like, the entire contents of which are hereby incorporated herein by reference. Except for application history documents that are inconsistent with or conflict with the contents of the present disclosure, the documents (currently or hereafter appended to the present disclosure) that limit the broadest scope of the claims of the present disclosure are also excluded. It should be noted that in the event of any inconsistency or conflict between the descriptions, definitions, and/or use of terms in the materials appended to the present disclosure and those set forth herein, the descriptions, definitions, and/or use of terms in the present disclosure shall prevail.

Finally, it should be understood that the embodiments described in the present disclosure are used only to illustrate the principles of the embodiments of the present disclosure. Other deformations may also fall within the scope of the present disclosure. As such, by way of example and not limitation, alternative configurations of embodiments of the present disclosure may be viewed as consistent with the teachings of the present disclosure. Correspondingly, the embodiments of the present disclosure are not limited to the embodiments expressly presented and described herein.

What is claimed is:

1. A dry powder inhaler, comprising:
a piercing base, the piercing base being provided with one or more piercing needles for piercing a capsule from a side;
an upper structure, the upper structure being formed with a capsule chamber and a piercing channel, wherein
the capsule chamber is configured for accommodating the capsule;
the piercing channel is arranged laterally outside of the capsule chamber and connected to the capsule chamber, and the piercing needles undergo a lateral piercing movement along the piercing channel;
a lower structure for accommodating a portion of the upper structure, the upper structure being subjected to a longitudinal linear movement relative to the lower structure; and
a drive structure arranged on at least one of the upper structure and the lower structure, the piercing base being driven by the drive structure to undergo movement; wherein
the drive structure moves with the upper structure and the lower structure undergoing a longitudinal linear relative approach movement drive structure drives the piercing base to move in a direction of the piercing channel, the piercing needles move with the piercing base along the piercing channel to pierce the capsule in the capsule chamber; the drive structure moves with the upper structure and the lower structure undergoing a longitudinal linear relative separation movement, the drive structure drives the piercing base to move in the direction of the piercing channel, and the piercing needles leave the capsule chamber with the piercing base along the piercing channel;
the piercing channel is a straight channel and the piercing needles are straight needles; and the piercing needles are contained in a portion of the piercing channel at any time; and
the drive structure includes an inclined track arranged within the lower structure for a tilting movement of the piercing base, the inclined track is tilted in a direction gradually inclined from a position at an upper edge of the lower structure to a lower portion of the lower structure and toward the capsule chamber, when the upper structure and the lower structure are subjected to the longitudinal linear relative approach movement, the piercing base is driven by the inclined track and guided by the piercing channel to move toward the capsule chamber, and the piercing needles enter the capsule chamber along the piercing channel to complete pierce of the capsule.

2. The dry powder inhaler according to claim 1, wherein
the direction of the piercing channel is horizontal, a length of the inclined track is L1, a piercing distance of the piercing needles is L2, and a longitudinal linear movement distance between the upper structure and the lower structure is L3, and a relationship between the length L1, the piercing distance L2, and the longitudinal linear movement distance L3 is represented that the length L1 is greater than a length of a hypotenuse of a right triangle formed by L2 and L3.

3. The dry powder inhaler according to claim 2, wherein the inclined track is groove-type inclined tracks provided on front and rear sidewalls of the lower structure, the piercing base is provided with protruding columns extending into the groove-type inclined tracks, and the piercing base is arranged around the capsule chamber; and
the inhaler is of single-side piercing type, two groove-type inclined tracks are provided on the front and rear sidewalls of the lower structure, the protruding columns extending into the two groove-type inclined tracks are provided on front and rear sides of the piercing base, the piercing base is provided with two piercing needles arranged in parallel, and two piercing channels arranged in parallel are provided on the same side in correspondence.

4. The dry powder inhaler according to claim 3, further comprising
a motion-guiding structure for ensuring movement of the piercing base along the piercing channel, the piercing base is provided with an adapting portion in combination with the motion-guiding structure, and the adapting portion moves on the motion-guiding structure;
wherein the motion-guiding structure is a cylindrical body or a columnar cavity extending from left and right sidewalls of the upper structure or the lower structure, and a direction of the motion-guiding structure is parallel to the piercing channel.

5. The dry powder inhaler according to claim 4, wherein the motion-guiding structure is the cylindrical body or the columnar cavity extending from a sidewall of the upper structure;
when the motion-guiding structure is the cylindrical body, the adapting portion is a columnar cavity sleeved on an outside of the cylindrical body, the protruding columns extend from an outside of the front and rear walls of the columnar cavity, or
when the motion-guiding structure is the columnar cavity, the adapting portion is a cylindrical body arranged inside the columnar cavity, the protruding columns extend from an outside of the front and rear walls of the cylindrical body, and transverse penetration notches are provided on the front and rear sidewalls of the columnar cavity to realize extending of the protruding columns and ensure protruding movement.

6. The dry powder inhaler according to claim 5, wherein secondary adaptation grooves are provided in the columnar cavity and secondary adaptation protrusions are provided on the cylindrical body, and the secondary adaptation grooves are provided symmetrically on an inside of the front and rear sidewalls or an inside of upper and lower sidewalls of the columnar cavity.

7. The dry powder inhaler according to claim 3, wherein a guide opening is provided at the uppermost portion of the lower structure, the guide opening is larger than an opening of the inclined track, and the guide opening is a triangular opening.

8. The dry powder inhaler according to claim 1, wherein the inclined track is a slope-type inclined track provided on left and right sidewalls of the lower structure, a movement track of the slope-type inclined track is an inclined surface of the slope-type inclined track, and the piercing base is provided with an anastomosing surface that fits into the inclined surface of the slope-type inclined track.

9. The dry powder inhaler according to claim 8, wherein
when the inhaler is of single-side piercing type, the inhaler includes a slope-type inclined track and the piercing base, wherein two piercing needles arranged in parallel are provided on the piercing base, and two piercing channels arranged in parallel are provided on the same side of the dry powder inhaler; or
when the inhaler is of double-side piercing type, two slope-type inclined tracks are symmetrically provided on the left and right sidewalls of the lower structure and two piercing bases corresponding to the two slope-type inclined tracks are provided, one piercing needle is provided on the piercing base, and two piercing channels arranged in parallel or in the same straight line are provided on both sides of the dry powder inhaler.

10. The dry powder inhaler according to claim 9, further comprising:
a fitting structure for fitting a main base on the inclined track, wherein
the fitting structure includes a spring structure providing elasticity on one side, one end of the spring structure being attached to a lower end of the upper structure and the other end of the spring structure being attached to a side of the main base; or
the fitting structure is a T-shaped limiting groove arranged on the inclined surface, and the fitting structure further includes an adapted T-shaped protrusion extending from the anastomosing surface.

11. The dry powder inhaler according to claim 1, wherein the lower structure is provided with an accommodating chamber, and the upper structure undergoes the longitudinal linear relative movement within the accommodating chamber of the lower structure,
in a stationary state, an anastomosis table shaped in line with an upper cavity of the lower structure is provided above the upper structure, an upper plane of the anastomosis table is horizontal to an upper plane of the upper cavity of the lower structure, the anastomosis table is perpendicularly in and out of the upper cavity, an operation table is provided above the anastomosis table, and finger placement structures are provided on both sides of the operation table; and
a distance between the anastomosis table and the operation table is not less than a longitudinal linear movement distance between the upper structure and the lower structure.

12. The dry powder inhaler according to claim 1, further comprising an anti-separation structure, wherein the anti-separation structure both ensures the longitudinal linear movement and prevents separation of the upper structure from the lower structure after the upper structure and the lower structure are combined; the anti-separation structure includes a limit rod extending from below the upper structure, a locking protrusion is provided at the lowermost part of the limit rod, a length of the limit rod is not less than a longitudinal linear movement distance between the upper structure and the lower structure, the lower structure is provided with a limit protrusion adapted to the locking protrusion, a position of the locking protrusion contacting the limit protrusion is an anti-separation position, and other positions except for the anti-separation position are positions for maintaining movement state.

13. The dry powder inhaler according to claim 12, wherein a length of the limit rod from the locking protrusion to the lowermost end of the upper structure is equal to the longitudinal linear movement distance between the upper structure and the lower structure.

14. The dry powder inhaler according to claim 12, wherein the limit protrusion is a protruding structure extending from a sidewall of the lower structure.

15. The dry powder inhaler according to claim 12, wherein the lower structure is provided with a movement groove for movement of the locking protrusion, the limit protrusion is a top body from a bottom of the movement groove to a sidewall of the lower structure, and the top body is provided at a top of the movement groove.

16. The dry powder inhaler according to claim 15, wherein a guide groove is provided above the movement groove, a direction and a width of the guide groove are identical to a direction and a width of the movement groove, a lowermost end of the guide groove is an uppermost part of the limit protrusion, and a guide surface is provided at the uppermost part of the limit protrusion.

17. The dry powder inhaler according to claim 12, wherein a reset structure is provided at a bottom of the upper structure and the lower structure to make the locking protrusion contact with the limit protrusion in an unused state, wherein the reset structure is a reset spring.

18. The dry powder inhaler according to claim 17, wherein mutually socketed lumen structures are provided at the bottom of the upper structure and at a bottom surface of the lower structure in correspondence, the lumen structures are an upper lumen at the bottom of the upper structure and a lower lumen extending from the bottom surface of the upper structure, the upper lumen is socketed on an outer side of the lower lumen or the upper lumen extends into an inner side of the lower lumen, and the reset spring is provided in a center of the upper lumen and the lower lumen; or
a bottom cavity for movement of a bottom structure of the upper structure is provided within the lower structure, the bottom structure is adapted to the bottom cavity in shape, the reset spring is provided in the bottom cavity, and a portion of the bottom structure is provided within the bottom cavity.

19. The dry powder inhaler according to claim 1, further comprising: a direction-guiding structure for maintaining the longitudinal linear movement between the upper structure and the lower structure, wherein the direction-guiding structure includes a direction-guiding groove or a direction-guiding column provided in a middle of the upper structure, the direction-guiding structure further includes a direction-guiding column or a direction-guiding groove provided in a middle of the lower structure in correspondence, the direction-guiding groove is adapted to the direction-guiding column, and directions of the direction-guiding groove and the direction-guiding column are in a longitudinal straight line.

20. A dry powder inhaler, comprising:
a piercing base, the piercing base being provided with one or more piercing needles for piercing a capsule from a side;
an upper structure, the upper structure being formed with a capsule chamber and a piercing channel, wherein
the capsule chamber is configured for accommodating the capsule;
the piercing channel is arranged laterally outside of the capsule chamber and connected to the capsule chamber, and the piercing needles undergo a lateral piercing movement along the piercing channel;
a lower structure for accommodating a portion of the upper structure, the upper structure being subjected to a longitudinal linear movement relative to the lower structure; and
a drive structure arranged on at least one of the upper structure and the lower structure, the piercing base being driven by the drive structure to undergo movement; wherein
the drive structure moves with the upper structure and the lower structure undergoing a longitudinal linear relative approach movement, the drive structure drives the piercing base to move in a direction of the piercing channel, the piercing needles move with the piercing base along the piercing channel to pierce the capsule in the capsule chamber; the drive structure moves with the upper structure and the lower structure undergoing a longitudinal linear relative separation movement, the drive structure drives the piercing base to move in the direction of the piercing channel, and the piercing needles leave the capsule chamber with the piercing base along the piercing channel;
the piercing channel is a straight channel and the piercing needles are straight needles; and the piercing needles are contained in a portion of the piercing channel at any time; and
the drive structure includes a linkage structure connected to the piercing base and a hinge structure; and the hinge structure is configured to articulate the linkage structure with the lower structure and the piercing base; wherein
the linkage structure includes at least one main connecting rod obliquely connected to the piercing base, one end of the main connecting rod is hinged to a sidewall of the lower structure through the hinge structure, and the other end of the main connecting rod is hinged to the piercing base through the hinge structure;
the main connecting rod is tilted in a direction gradually tilting upwardly from a position at the upper edge of the lower structure and toward the capsule chamber; and
when the upper structure and the lower structure undergo the longitudinal linear relative movement, the main connecting rod rotates with the longitudinal linear relative movement, the main connecting rod rotates to drive the piercing base to move in the direction of the piercing channel, and the piercing needles enter the capsule chamber along the piercing channel with the piercing base to pierce the capsule in the capsule chamber.
